(19) United States
(12) Patent Application Publication
(10) Pub. No.: US 2026/0347348 A1
(43) Pub. Date: Nov. 5, 2026

(54) JAK1/JAK2/TYK2 Inhibitors For Topical Treatment of Dermatological Diseases

(71) Applicant: Hangzhou Highlightll Pharmaceutical Co., Ltd, Hangzhou (CN)

(72) Inventors: Congxin Liang, Hangzhou (CN); Shuangjiang Li, Hangzhou (CN); Xiaojing Tang, Hangzhou (CN); Junmin Huang, Princeton, NJ (US)

(73) Assignee: Hangzhou Highlightll Pharmaceutical Co., Ltd, Hangzhou (CN)

(21) Appl. No.: 19/104,582

(22) PCT Filed: Aug. 10, 2023
(86) PCT No.: PCT/CN2023/112361

§ 371 Date: Feb. 18, 2025

(30) Foreign Application Priority Data
Aug. 22, 2022 (WO) PCT/CN2022/113873

(51) Int. Cl. C07D 413/14; A61K 31/506; A61K 31/5377; A61P 17/00; A61P 17/06; C07D 401/14; C07D 403/12; C07D 403/14; C07D 405/14

(52) U.S. Cl. CPC C07D 413/14; A61K 31/506; A61K 31/5377; A61P 17/00; A61P 17/06; C07D 401/14; C07D 403/12; C07D 403/14; C07D 405/14

(57) ABSTRACT

Provided 4-pyrazolyl-N-heteroarylpyrimidin-2-amine compounds as potent TYK2/JAK1/JAK2 inhibitors with low systemic exposures when applied topically. Thus, these compounds, and compositions are useful as topical treatment of dermatological disorders related to JAK1/JAK2/TYK2 such as atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus.

### BACKGROUND

The Janus kinase family comprises 4 known family members: JAK 1, 2, 3, and tyrosine kinase 2 (TYK2). These cytoplasmic tyrosine kinases are associated with membrane cytokine receptors such as common gamma-chain receptors and the glycoprotein 130 (gp 130) trans-membrane proteins (Murray, J. Immunol. 178(5):2623-2629, 2007). Almost 40 cytokine receptors signal through combinations of these 4 JAK family members and their 7 downstream substrates: the signal transduction activators of transcription (STAT) family members (Ghoreschi et al., Immunol Rev. 228(1):273-287, 2009). Cytokine binding to its receptor initiates JAK activation via trans- and auto-phosphorylation. The JAK family kinases in turn phosphorylate cytokine receptor residues, creating binding sites for sarcoma homology 2 (SH2) containing proteins, such as the STAT factors and other regulators, which are subsequently activated by JAK phosphorylation. Activated STATs enter the nucleus initiating expression of survival factors, cytokines, chemokines, and molecules that facilitate leukocyte cellular trafficking (Schindler et al., J. Biol. Chem. 282(28):20059-20063, 2007). JAK activation also results in cell proliferation via phosphoinositide 3-kinase (PI3K) and protein kinase B-mediated pathways.

Tofacitinib, a JAK1/JAK3/JAK2 inhibitor, was the first JAK inhibitor approved by FDA for the treatment of rheumatoid arthritis, psoriatic arthritis, ankylosing spondylitis, ulcerative colitis. Recently, JAK1 selective compounds, upadacitinib and abrocitinib, have been approved for the treatment of atopic dermatitis (Nakashima et al. Allergol Int. 2022 January; 71(1):40-46). TYK2 selective inhibitor deucravacitinib has shown positive Phase 2 and Phase 3 results in patients with psoriasis (Papp et al. N Engl J Med. 2018, 379; 14), and is approved by FDA for the treatment of psoriasis. The topical formulation of ruxolitinib (Opzelura™), a selective JAK1/JAK2 inhibitor, is also approved for the treatment of mild to moderate atopic dermatitis, and is in clinical development for the treatment of psoriasis, alopecia areata, vitiligo, hidradenitis suppurativa, hand eczema, necrobiosis lipoidica, non-sclerotic cutaneous chronic graft-versus-host disease, hand dermatitis, and Lichen planus. Thus, JAK inhibitors are useful in treating various dermatological diseases such as atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus (Zhang et al. J Inflamm Res. 2022 Mar. 18; 15:1935-1941).

However, the approved oral agents, upadacitinib and abrocitinib, carry black box warnings of risks such as serious infections, non-melanoma skin cancers, thrombosis, and thrombocytopenia. Interestingly, the topical formulation of ruxolitinib also carry similar risks, probably because its systemic exposure is also high, with Cmax reaching 449±883 nM when applied, topically, 1.2-37.6 g per application, twice a day (Medication Guide approved by FDA, September 2021), well above its IC₅₀ for JAK1 and JAK2 inhibition in biochemical assay (3.3 nM and 2.8 nM respectively) or inhibition of JAK1, JAK2 related cellular assays (maximum effect at −300 nM, ruxolitinib FDA pharmacology review 2011).

Many of the reported JAK inhibitors, such as tofacitinib, abrocitinib, ruxolitinib, baricitinib, oclacitinib, share the pyrrolopyrimidine core structure as illustrated below:

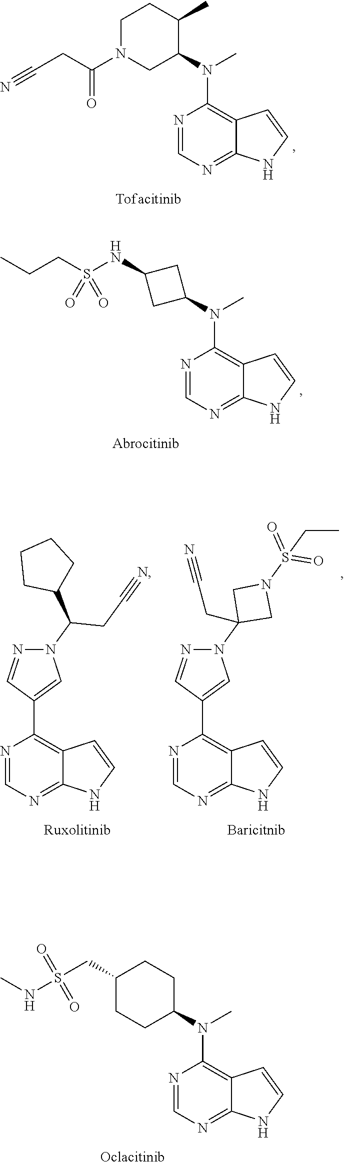 [SMILES: C[C@@H]1CCN(C(=O)CC#N)C[C@@H]1N(C)c1ncnc2[nH]ccc12 ; CCCS(=O)(=O)N[C@H]1C[C@@H](N(C)c2ncnc3[nH]ccc23)C1 ; N#CC[C@H](C1CCCC1)n1cc(-c2ncnc3[nH]ccc23)cn1 ; CCS(=O)(=O)N1CC(CC#N)(n2cc(-c3ncnc4[nH]ccc34)cn2)C1 ; "," ; CNS(=O)(=O)C[C@H]1CC[C@H](N(C)c2ncnc3[nH]ccc23)CC1]

WO2009/064835 disclosed compounds represented by D1 and D2 as JAK2 inhibitors. WO2012/062704 and WO2020/119819 disclosed compounds as shown in D3 as JAK1/JAK2/TYK2 inhibitors. WO2014/111037 disclosed compounds represented by D4 as JAK1/JAK2/JAK3 inhibitors. WO2021/078022 disclosed compounds represented by D5 as JAK1/JAK2/TYK2 inhibitors.

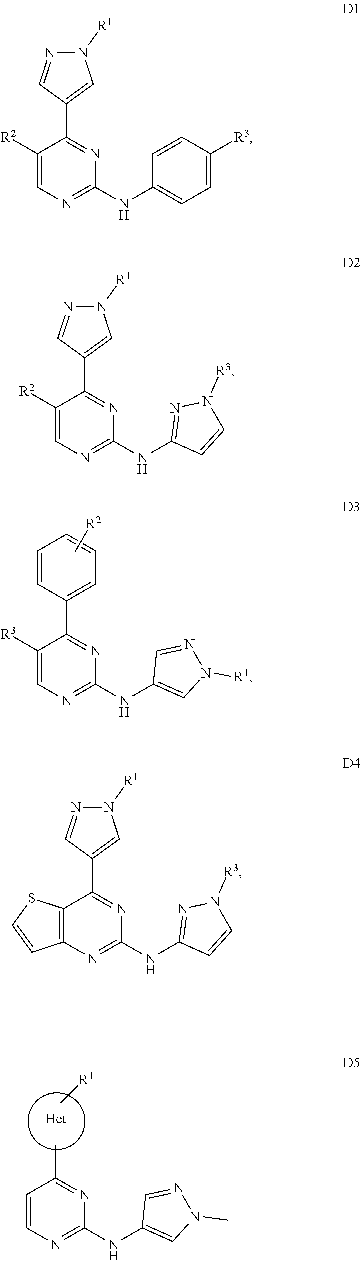

This invention discloses novel 4-pyrazolyl-N-heteroarylpyrimidin-2-amine compounds as potent TYK2/JAK1/JAK2 inhibitors with low systemic exposures when applied topically. Thus, these compounds, and compositions comprising a compound of this disclosure are useful as topical treatment of dermatological disorders related to JAK1/JAK2/TYK2 such as atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus.

### SUMMARY

The present disclosure provides novel 4-pyrazolyl-N-heteroarylpyrimidin-2-amine compounds, or salts, solvates, hydrates, polymorphs, co-crystals, tautomers, stereoisomers, isotopically labeled derivatives, or prodrugs thereof, as TYK2/JAK1/JAK2 kinase inhibitors that can be quickly degraded by liver microsomes, and as such advantageous properties suited as a topical agent for use in treating dermatological disorders. The present disclosure also provides compositions comprising a compound of this disclosure or a salt, solvate, hydrate, polymorph, co-crystal, tautomer, stereoisomer, isotopically labeled derivative, or prodrug thereof. The present disclosure further provides methods of use of such compounds, or salts, solvates, hydrates, polymorphs, co-crystals, tautomers, stereoisomers, isotopically labeled derivatives, or prodrugs thereof, or such compositions, for treating disorders associated with TYK2, JAK1, JAK2, including dermatological diseases such as atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus.

In one aspect, provided is a compound of Formula (I):

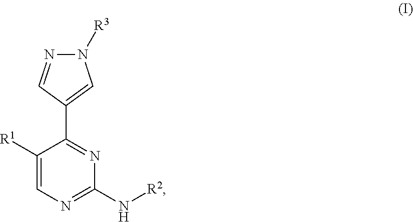

or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is

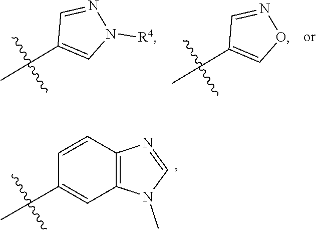

wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In another aspect, provided is a compound of Formula (II):

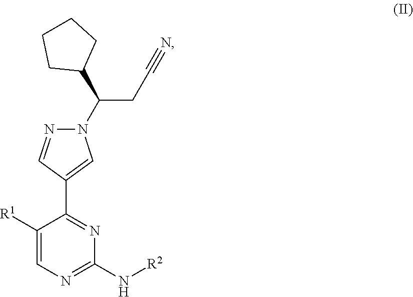

or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is

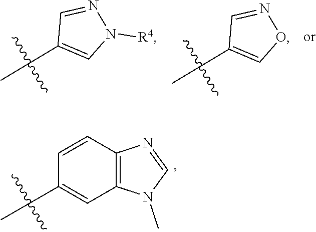

wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′; R, R′ each is independently H, or C₁₋₃ alkyl optionally substituted with 1, 2, or 3 substituents independently selected from the group consisting of halo, OH, and CN.

In another aspect, provided is a compound of Formula (III):

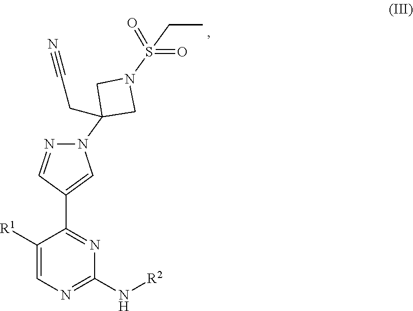

or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is

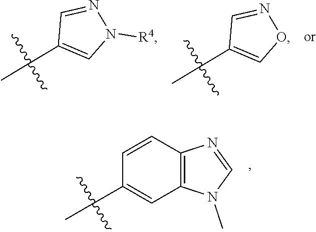

wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′; R, R′ each is independently H, or C₁₋₃ alkyl optionally substituted with 1, 2, or 3 substituents independently selected from the group consisting of halo, OH, and CN.

The compounds of this invention, and compositions comprising them, are useful for treating or lessening the severity of TYK2, JAK1, and JAK2 modulated diseases, disorders, or symptoms thereof.

Accordingly, one aspect of the present disclosure relates to a method for treating a dermatological disorder, the method comprising administering to a subject in need thereof an effective amount of a compound of Formulae (I)-(III) herein, or a salt, solvate, hydrate, polymorph, co-crystal, tautomer, stereoisomer, isotopically labeled derivative, or prodrug thereof, which may be formulated into a composition. In some examples, the composition may be a pharmaceutical composition, which may further comprise a pharmaceutically acceptable carrier.

In some embodiments, the dermatological disorder is atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus.

In some embodiments, the dermatological disorder can be any of those modulated by TYK2 JAK1 and JAK2. In certain embodiments, the dermatological disorder is atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus.

Also within the scope of the present disclosure are: (i) pharmaceutical compositions for use in treating the target dermatological disorders as described herein, the pharmaceutical composition comprising a compound of Formulae (I)-(III), or salt, solvate, hydrate, polymorph, co-crystal, tautomer, stereoisomer, isotopically labeled derivative, or prodrug thereof, and a pharmaceutically acceptable carrier; and (ii) uses of a compound of Formulae (I)-(III), or salt, solvate, hydrate, polymorph, co-crystal, tautomer, stereoisomer, isotopically labeled derivative, or prodrug thereof, for manufacturing a medicament for use in treating any of the target dermatological disorders.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

The details of one or more embodiments of the invention are set forth in the description below. Other features or advantages of the present invention will be apparent from the following drawings and detailed description of several embodiments, and also from the appended claims.

### Definitions

Definitions of specific chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds described herein, even though only a single tautomeric form may be represented. All such isomeric forms of such compounds herein are expressly included in the present disclosure. The term “isomers” is intended to include diastereoisomers, enantiomers, regioisomers, structural isomers, rotational isomers, tautomers, and the like. For compounds which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disorder or a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disorder or disease described herein. In some embodiments, treatment may be administered after one or more signs or symptoms of the disease have developed or have been observed. In other embodiments, treatment may be administered in the absence of signs or symptoms of the disorder. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably. An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for TYK2/JAK1/JAK2 kinase inhibition. In certain embodiments, a therapeutically effective amount is an amount sufficient for treating a dermatological disorder. In certain embodiments, a therapeutically effective amount is an amount sufficient for TYK2/JAK1/JAK2 kinase inhibition and treating a dermatological disorder.

### DETAILED DESCRIPTION OF THE INVENTION

The present disclosure is based at least in part on the unexpected results that compounds of Formulae (I)-(III) are selective TYK2/JAK1/JAK2 kinase inhibitors that have a good exposure in skin but low in plasma.

In one aspect, provided is a compound of Formula (I):

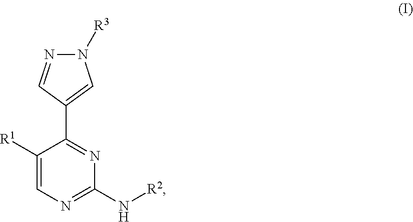

or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is

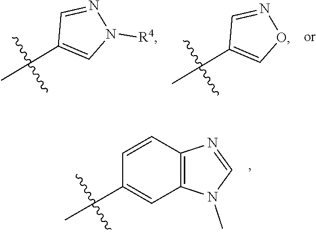

wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In another aspect, provided is a compound of Formula (II):

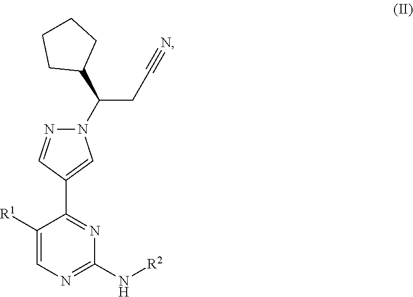

or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is

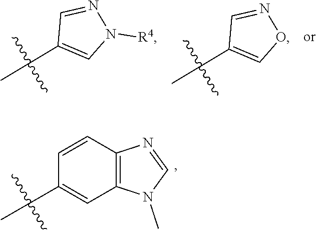

wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In another aspect, provided is a compound of Formula (III):

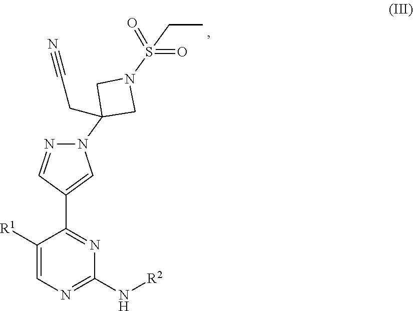

or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is

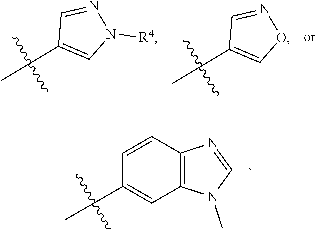

wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In some embodiments, the invention provides a compound of any one of the Formulae (I)-(III), or a pharmaceutically acceptable salt thereof, wherein: R² is

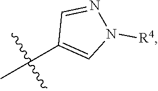

wherein R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In some embodiments, the invention provides a compound of any one of the Formulae (I)-(III), or a pharmaceutically acceptable salt thereof, wherein: R² is

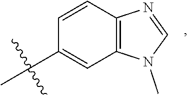 [SMILES: *c1ccc2ncn(C)c2c1]

In some embodiments, the invention provides a compound of any one of the Formulae (I)-(III), or a pharmaceutically acceptable salt thereof, wherein: R² is

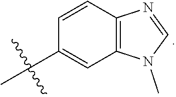 [SMILES: *c1ccc2ncn(C)c2c1]

In some embodiments, the invention provides a compound of any one of the Formulae (I)-(III), or a pharmaceutically acceptable salt thereof, wherein: R¹ is C₁₋₃ alky, C₃₋₄ cycloalkyl, or halo.

In some embodiments, the invention provides a compound of any one of the Formulae (I)-(III), or a pharmaceutically acceptable salt thereof, wherein: R¹ is C₁₋₃ alky.

In some embodiments, the invention provides a compound of any one of the Formulae (I)-(III), or a pharmaceutically acceptable salt thereof, wherein: R¹ is CH₃.

In some embodiments, the invention provides a compound of any one of the Formulae (I)-(III), or a pharmaceutically acceptable salt thereof, wherein: R¹ is C₃₋₄ cycloalkyl.

In some embodiments, the invention provides a compound of any one of the Formulae (I)-(III), or a pharmaceutically acceptable salt thereof, wherein: R¹ is cyclopropyl.

In some embodiments, the invention provides a compound of any one of the Formulae (I)-(III), or a pharmaceutically acceptable salt thereof, wherein: R¹ is halo.

In some embodiments, the invention provides a compound of any one of the Formulae (I)-(III), or a pharmaceutically acceptable salt thereof, wherein: R¹ is Cl.

In some embodiments, the invention provides a compound of any one of the Formulae (I)-(III), or a pharmaceutically acceptable salt thereof, wherein:
R⁴ is C₁₋₃ alkyl, optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In some embodiments, the invention provides a compound of any one of the Formulae (I)-(III), or a pharmaceutically acceptable salt thereof, wherein: R⁴ is CH₂CH₂OH.

In some embodiments, the invention provides a compound of any one of the Formulae (I)-(III), or a pharmaceutically acceptable salt thereof, wherein: R⁴ is isopropyl.

In some embodiments, the invention provides a compound of any one of the Formulae (I)-(III), or a pharmaceutically acceptable salt thereof, wherein: R⁴ is CH₃.

In some embodiments, the invention provides a compound of any one of the Formulae (I)-(III), or a pharmaceutically acceptable salt thereof, wherein: R⁴ is CH₂R⁵, R⁵ is 4 to 6 membered heterocyclyl.

In some embodiments, the invention provides a compound of any one of the Formulae (I)-(III), or a pharmaceutically acceptable salt thereof, wherein:
R⁴ is CH₂R⁵, R⁵ is

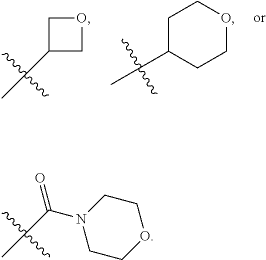 [SMILES: *C1COC1 ; *C1CCOCC1 ; "or" ; *C(=O)N1CCOCC1]

In some embodiments, the invention provides a compound of any one of the Formulae (I)-(III), or a pharmaceutically acceptable salt thereof, wherein:
R⁴ is 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

In some embodiments, the invention provides a compound of any one of the Formulae (I)-(III), or a pharmaceutically acceptable salt thereof, wherein: R⁴ is

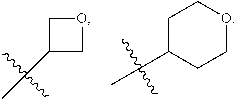 [SMILES: *C1COC1 ; *C1CCOCC1]

In some embodiments, the invention provides a compound of any one of the Formulae (I)-(III), or a pharmaceutically acceptable salt thereof, wherein the compound is depicted in Table 1: Table 1:

TABLE 1 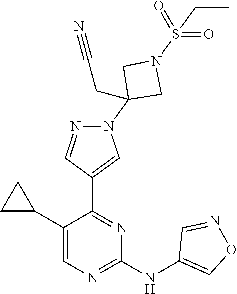 [SMILES: CCS(=O)(=O)N1CC(CC#N)(n2cc(-c3nc(Nc4cnoc4)ncc3C3CC3)cn2)C1]
1 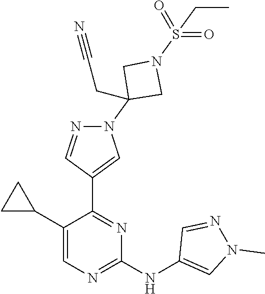 [SMILES: CCS(=O)(=O)N1CC(CC#N)(n2cc(-c3nc(Nc4cnn(C)c4)ncc3C3CC3)cn2)C1]
2 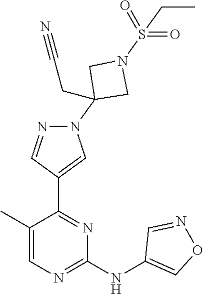 [SMILES: CCS(=O)(=O)N1CC(CC#N)(n2cc(-c3nc(Nc4cnoc4)ncc3C)cn2)C1]
3 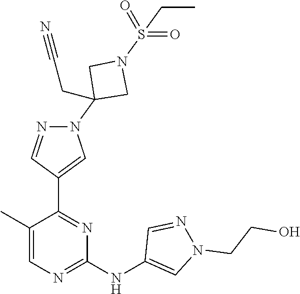 [SMILES: CCS(=O)(=O)N1CC(CC#N)(n2cc(-c3nc(Nc4cnn(CCO)c4)ncc3C)cn2)C1]
12 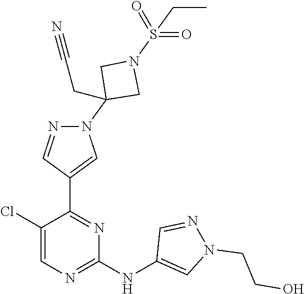 [SMILES: CCS(=O)(=O)N1CC(CC#N)(n2cc(-c3nc(Nc4cnn(CCO)c4)ncc3Cl)cn2)C1]
13 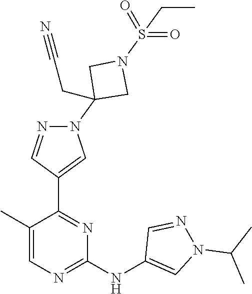 [SMILES: CCS(=O)(=O)N1CC(CC#N)(n2cc(-c3nc(Nc4cnn(C(C)C)c4)ncc3C)cn2)C1]
17 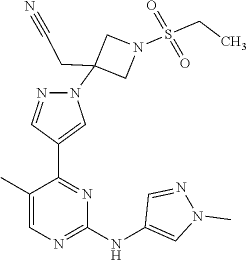
18 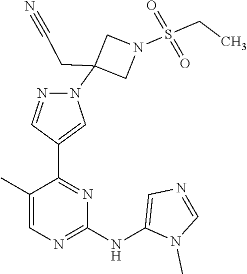 [SMILES: CCS(=O)(=O)N1CC(CC#N)(n2cc(-c3nc(Nc4cncn4C)ncc3C)cn2)C1]
26 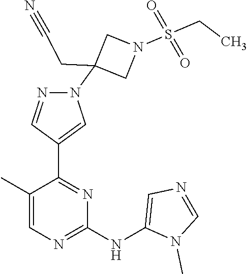 [SMILES: CCS(=O)(=O)N1CC(CC#N)(n2cc(-c3nc(Nc4cncn4C)ncc3C)cn2)C1]
27

Representative compounds of the invention are listed below:
2-(3-(4-(5-cyclopropyl-2-(isoxazol-4-ylamino)pyrimidin-4-yl)-1H-pyrazol-1-yl)-1-(ethylsulfonyl) azetidin-3-yl) acetonitrile (1);
2-(3-(4-(5-cyclopropyl-2-((1-methyl-1H-pyrazol-4-yl) amino) pyrimidin-4-yl)-1H-pyrazol-1-yl)-1-(ethylsulfonyl) azetidin-3-yl) acetonitrile (2);
2-(1-(ethylsulfonyl)-3-(4-(2-(isoxazol-4-ylamino)-5-methylpyrimidin-4-yl)-1H-pyrazol-1-yl) azetidin-3-yl) acetonitrile (3);
2-(1-(ethylsulfonyl)-3-(4-(2-((1-(2-hydroxyethyl)-1H-pyrazol-4-yl)amino)-5-methylpyrimidin-4-yl)-1H-pyrazol-1-yl)azetidin-3-yl)acetonitrile (12);
2-(3-(4-(5-chloro-2-((1-(2-hydroxyethyl)-1H-pyrazol-4-yl)amino)pyrimidin-4-yl)-1H-pyrazol-1-yl)-1-(ethylsulfonyl)azetidin-3-yl)acetonitrile (13);
2-(1-(ethylsulfonyl)-3-(4-(2-((1-isopropyl-1H-pyrazol-4-yl)amino)-5-methylpyrimidin-4-yl)-1H-pyrazol-1-yl)azetidin-3-yl)acetonitrile (17);
2-(1-(ethylsulfonyl)-3-(4-(5-methyl-2-((1-methyl-1H-pyrazol-4-yl)amino)pyrimidin-4-yl)-1H-pyrazol-1-yl)azetidin-3-yl)acetonitrile (18);
2-(1-(ethylsulfonyl)-3-(4-(5-methyl-2-((1-methyl-1H-benzo[d]imidazol-6-yl)amino)pyrimidin-4-yl)-1H-pyrazol-1-yl)azetidin-3-yl)acetonitrile (26);
2-(3-(4-(5-chloro-2-((1-methyl-1H-benzo[d]imidazol-6-yl)amino)pyrimidin-4-yl)-1H-pyrazol-1-yl)-1-(ethylsulfonyl)azetidin-3-yl)acetonitrile (27).

In some embodiments, the invention provides a compound of any one of the Formulae (I)-(III), or a pharmaceutically acceptable salt thereof, wherein the compound is depicted in Table 2:

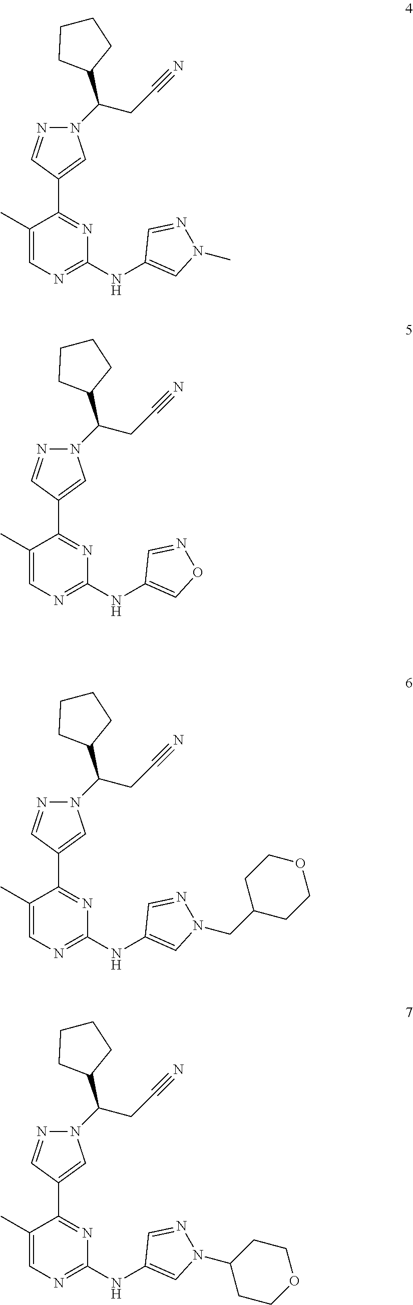

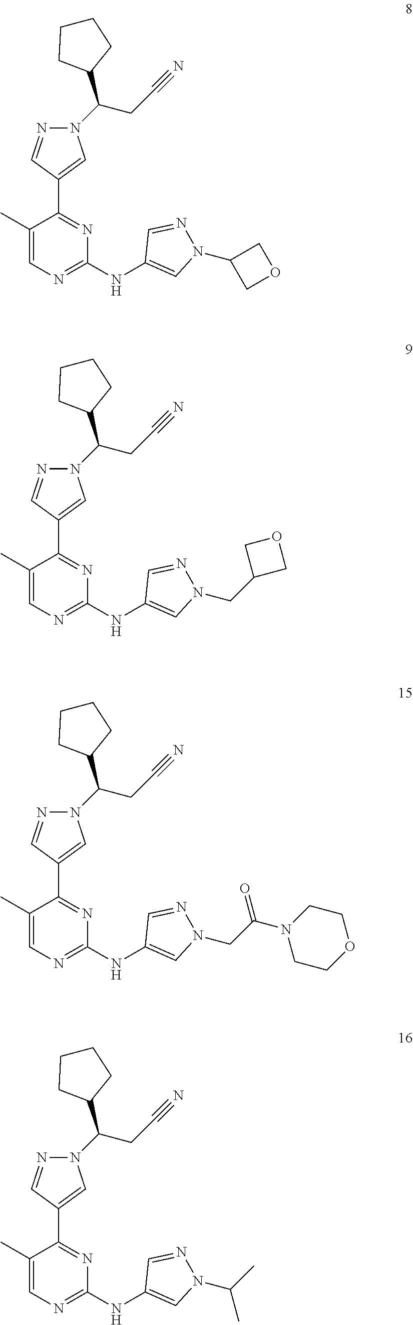 [SMILES: Cc1cnc(Nc2cnn(C3COC3)c2)nc1-c1cnn([C@H](CC#N)C2CCCC2)c1 ; Cc1cnc(Nc2cnn(CC3COC3)c2)nc1-c1cnn([C@H](CC#N)C2CCCC2)c1 ; Cc1cnc(Nc2cnn(CC(=O)N3CCOCC3)c2)nc1-c1cnn([C@H](CC#N)C2CCCC2)c1 ; Cc1cnc(Nc2cnn(C(C)C)c2)nc1-c1cnn([C@H](CC#N)C2CCCC2)c1]

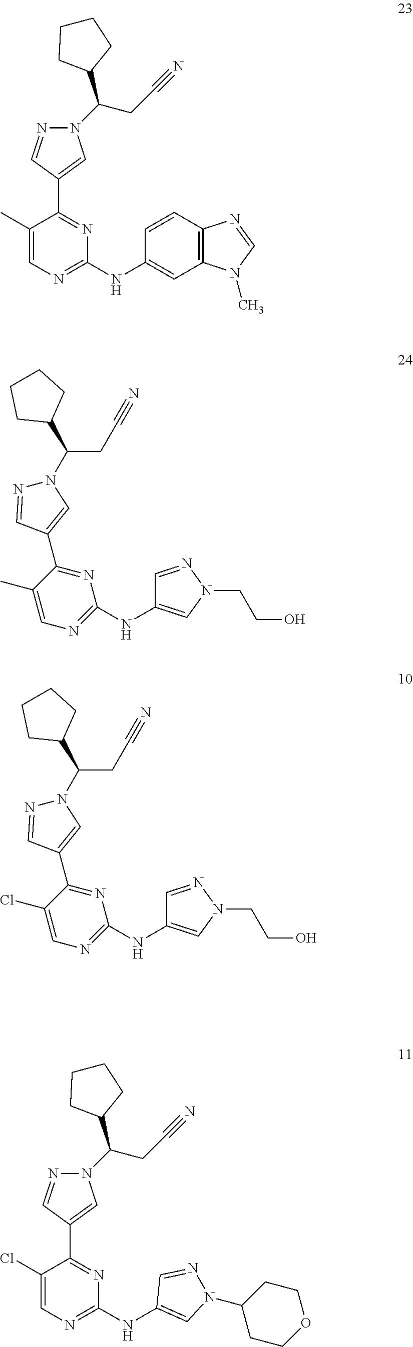 [SMILES: Cc1cnc(Nc2ccc3ncn(C)c3c2)nc1-c1cnn([C@H](CC#N)C2CCCC2)c1 ; Cc1cnc(Nc2cnn(CCO)c2)nc1-c1cnn([C@H](CC#N)C2CCCC2)c1 ; N#CC[C@H](C1CCCC1)n1cc(-c2nc(Nc3cnn(CCO)c3)ncc2Cl)cn1 ; N#CC[C@H](C1CCCC1)n1cc(-c2nc(Nc3cnn(C4CCOCC4)c3)ncc2Cl)cn1]

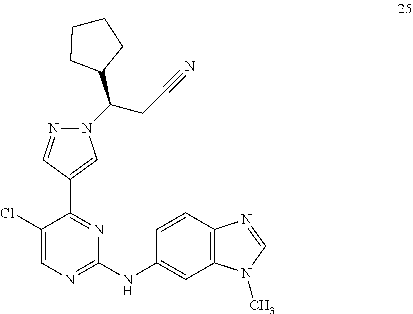 [SMILES: Cn1cnc2ccc(Nc3ncc(Cl)c(-c4cnn([C@H](CC#N)C5CCCC5)c4)n3)cc21]

Representative compounds of the invention are listed below:
(R)-3-cyclopentyl-3-(4-(5-methyl-2-((1-methyl-1H-pyrazol-4-yl) amino) pyrimidin-4-yl)-1H-pyrazol-1-yl) propanenitrile (4);
(R)-3-cyclopentyl-3-(4-(2-(isoxazol-4-ylamino)-5-methylpyrimidin-4-yl)-1H-pyrazol-1-yl) propanenitrile (5);
(R)-3-cyclopentyl-3-(4-(5-methyl-2-((1-((tetrahydro-2H-pyran-4-yl) methyl)-1H-pyrazol-4-yl) amino) pyrimidin-4-yl)-1H-pyrazol-1-yl)propanenitrile (6);
(R)-3-cyclopentyl-3-(4-(5-methyl-2-((1-(tetrahydro-2H-pyran-4-yl)-1H-pyrazol-4-yl) amino) pyrimidin-4-yl)-1H-pyrazol-1-yl)propanenitrile (7);
(R)-3-cyclopentyl-3-(4-(5-methyl-2-((1-(oxetan-3-yl)-1H-pyrazol-4-yl) amino) pyrimidin-4-yl)-1H-pyrazol-1-yl) propanenitrile (8);
(R)-3-cyclopentyl-3-(4-(5-methyl-2-((1-(oxetan-3-ylmethyl)-1H-pyrazol-4-yl) amino) pyrimidin-4-yl)-1H-pyrazol-1-yl) propanenitrile (9);
(R)-3-(4-(5-chloro-2-((1-(2-hydroxyethyl)-1H-pyrazol-4-yl)amino)pyrimidin-4-yl)-1H-pyrazol-1-yl)-3-cyclopentylpropanenitrile (10);
((R)-3-(4-(5-chloro-2-((1-(tetrahydro-2H-pyran-4-yl)-1H-pyrazol-4-yl)amino)pyrimidin-4-yl)-1H-pyrazol-1-yl)-3-cyclopentylpropanenitrile (11);
(R)-3-cyclopentyl-3-(4-(5-methyl-2-((1-(2-morpholino-2-oxoethyl)-1H-pyrazol-4-yl)amino)pyrimidin-4-yl)-1H-pyrazol-1-yl)propanenitrile (15);
(R)-3-cyclopentyl-3-(4-(2-((1-isopropyl-1H-pyrazol-4-yl)amino)-5-methylpyrimidin-4-yl)-1H-pyrazol-1-yl)propanenitrile (16);
(R)-3-cyclopentyl-3-(4-(5-methyl-2-((1-methyl-1H-benzo[d]imidazol-6-yl)amino)pyrimidin-4-yl)-1H-pyrazol-1-yl)propanenitrile (23);
(R)-3-cyclopentyl-3-(4-(2-((1-(2-hydroxyethyl)-1H-pyrazol-4-yl)amino)-5-methylpyrimidin-4-yl)-1H-pyrazol-1-yl)propanenitrile (24);
(R)-3-(4-(5-chloro-2-((1-methyl-1H-benzo[d]imidazol-6-yl)amino)pyrimidin-4-yl)-1H-pyrazol-1-yl)-3-cyclopentylpropanenitrile (25).

In some embodiments, the invention provides a compound of any one of the Formulae (I)-(III), or a pharmaceutically acceptable salt thereof, wherein the compound is: (R)-3-cyclopentyl-3-(4-(5-methyl-2-((1-methyl-1H-pyrazol-4-yl) amino) pyrimidin-4-yl)-1H-pyrazol-1-yl) propanenitrile (4);

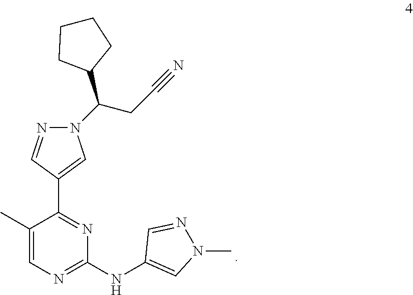

In some embodiments, the invention provides a compound of any one of the Formulae (I)-(III), or a pharmaceutically acceptable salt thereof, wherein the compound is: (R)-3-cyclopentyl-3-(4-(5-methyl-2-((1-methyl-1H-benzo[d]imidazol-6-yl)amino)pyrimidin-4-yl)-1H-pyrazol-1-yl)propanenitrile (23);

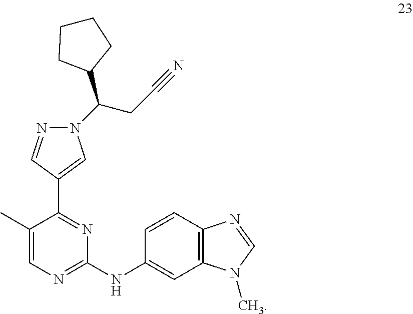 [SMILES: Cc1cnc(Nc2ccc3ncn(C)c3c2)nc1-c1cnn([C@H](CC#N)C2CCCC2)c1]

In some embodiments, the invention provides a compound of any one of the Formulae (I)-(III), or a pharmaceutically acceptable salt thereof, wherein the compound is: (R)-3-cyclopentyl-3-(4-(2-((1-(2-hydroxyethyl)-1H-pyrazol-4-yl)amino)-5-methylpyrimidin-4-yl)-1H-pyrazol-1-yl)propanenitrile (24);

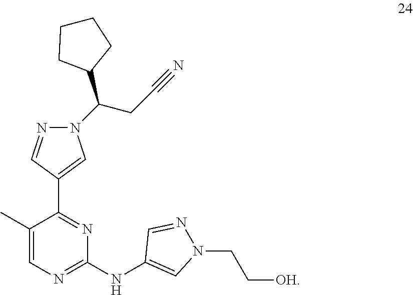 [SMILES: Cc1cnc(Nc2cnn(CCO)c2)nc1-c1cnn([C@H](CC#N)C2CCCC2)c1]

In another aspect, the invention provides a crystal Form A of compound (4),

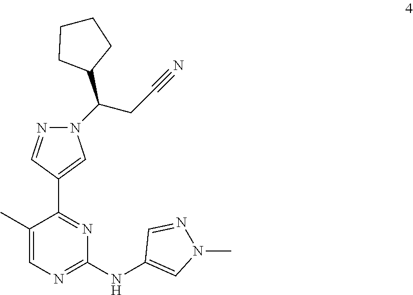

wherein the X-ray powder diffraction pattern of the crystal form shows characteristic peaks at 2theta angles of 10.3°±0.2°, 14.5°±0.2°, and 16.7°±0.2°.

In some embodiments, the invention provides a crystal Form A of compound (4), wherein the X-ray powder diffraction pattern of the crystal form shows characteristic peaks at 2theta angles of 10.3°±0.2°, 14.5°±0.2°, 16.7°±0.2°, 19.8°±0.2°, 23.3°±0.2°, and 23.5°±0.2°.

In some embodiments, the invention provides a crystal Form A of compound (4), wherein the X-ray powder diffraction pattern of the crystal form shows characteristic peaks at 2theta angles of 10.3°±0.2°, 14.5°±0.2°, 16.7°+0.2°, 19.8°±0.2°, 20.7°±0.2°, 23.3°±0.2°, 23.5°±0.2°, 27.2° 0.2°, and 28.10°±0.2°.

Accordingly, described herein are methods for treating dermatological disorders such as those described herein using an effective amount of a compound of Formulae (I)-(III), or salt, solvate, hydrate, polymorph, co-crystal, tautomer, stereoisomer, isotopically labeled derivative, or prodrug thereof, or a composition comprising a compound of Formulae (I)-(III), or salt, solvate, hydrate, polymorph, co-crystal, tautomer, stereoisomer, isotopically labeled derivative, or prodrug thereof.

### BRIEF DESCRIPTION OF THE FIGURES

FIG. 1: Ear thickness in hIL-23 induced psoriasis model for compound 4.

FIG. 2: Ear thickness in DNFB induced atopic dermatitis model for compound 4.

FIG. 3: XRPD for Form A of compound 4.

FIG. 4: TGA and DSC curve of Form A of compound 4.

### COMPOSITIONS

The present disclosure provides pharmaceutical compositions comprising a compound of Formulae (I)-(III), or a salt, solvate, hydrate, polymorph, co-crystal, tautomer, stereoisomer, isotopically labeled derivative, or prodrug thereof, and optionally a pharmaceutically acceptable excipient. In certain embodiments, the pharmaceutical composition described herein comprises a compound of Formulae (I)-(III), or a salt, solvate, hydrate, polymorph, co-crystal, tautomer, stereoisomer, isotopically labeled derivative, or prodrug thereof, and a pharmaceutically acceptable excipient.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, PA (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as those herein and other compounds known in the art, are known in the art and described in several issued US Patents, some of which include, but are not limited to, U.S. Pat. Nos. 4,369,172; and 4,842,866, and references cited therein. Coatings can be used for delivery of compounds to the intestine (see, e.g., U.S. Pat. Nos. 6,638,534, 5,217,720, and 6,569,457, 6,461,631, 6,528,080, 6,800,663, and references cited therein). A useful formulation for the compounds of this invention is the form of enteric pellets of which the enteric layer comprises hydroxypropylmethylcellulose acetate succinate.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or central nervous system) relative to the parent species. Preferred prodrugs include derivatives where a group that enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs’, Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development, Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development, 2 ed.; Overseas Publ.: Amsterdam, 1996; pp 351-385; Pitman, I. H. Medicinal Research Reviews 1981, 1, 189-214.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

In another embodiment, a composition of the present invention further comprises a second therapeutic agent. The second therapeutic agent includes any compound or therapeutic agent known to have or that demonstrates advantageous properties when administered alone or with a compound of any of the formulae herein. Drugs that could be usefully combined with these compounds include other kinase inhibitors and/or other therapeutic agents for the treatment of the diseases and disorders discussed above.

Such agents are described in detail in the art. Preferably, the second therapeutic agent is an agent useful in the treatment or prevention of a disease or condition selected from cancer and neoplastic diseases or disorders, autoimmune and inflammatory diseases or disorders, neurodegenerative disease or disorders.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et ah, (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of a compound of this invention can range from about 0.001 mg/kg to about 500 mg/kg, more preferably 0.01 mg/kg to about 50 mg/kg, more preferably 0.1 mg/kg to about 2.5 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

### Methods of Treatment

As generally described herein, the present disclosure provides methods of treating a dermatological disorder (or symptoms thereof) comprising administering an effective amount of a compound of the present disclosure, or a salt, solvate, hydrate, polymorph, co-crystal, tautomer, stereoisomer, isotopically labeled derivative, or prodrug thereof, or a composition comprising an effective amount of a compound of the present disclosure, or a salt, solvate, hydrate, polymorph, co-crystal, tautomer, stereoisomer, isotopically labeled derivative, or prodrug thereof, to a subject in need thereof. Such a method can be conducted in vivo (i.e., by administration to a subject) or in vitro (e.g., upon contact with a tissue or cell culture). Treating, as used herein, encompasses therapeutic treatment. In certain embodiments, the subject is identified as in need thereof. In certain embodiments, the method further comprises wherein the subject is treated; that is, the disease, disorder, or symptom thereof is ameliorated.

In certain embodiments, the effective amount is a therapeutically effective amount. For example, in certain embodiments, the method slows the progress of a dermatological disorder in the subject. In certain embodiments, the method improves the condition of the subject suffering from a dermatological disorder. In certain embodiments, the subject has a suspected or confirmed dermatological disorder.

Exemplary dermatological disorders include, but are not limited to, atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus.

In certain embodiments, the dermatological disorder is modulated by the JAK-STAT pathway. In certain embodiments, the dermatological disorder is modulated by JAK1. In certain embodiments, the dermatological disorder is modulated by JAK2. In certain embodiments, the dermatological disorder is modulated by TYK2. In certain embodiments, the dermatological disorder is modulated by any combination of TYK2, JAK1, and JAK2.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof, which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

Unless otherwise noted, reagents and solvents were used as received from commercial suppliers.

Temperatures are given in degrees Celsius. If not mentioned otherwise, all evaporations are performed under reduced pressure. The structure of final products, intermediates and starting materials is confirmed by standard analytical methods, e.g., microanalysis and spectroscopic characteristics, e.g., MS, IR, NMR. Abbreviations used are those conventional in the art.

### Example 1: Preparation of 2-(3-(4-(5-cyclopropyl-2-(isoxazol-4-ylamino) pyrimidin-4-yl)-1H-pyrazol-1-yl)-1-(ethylsulfonyl) azetidin-3-yl) acetonitrile (1)

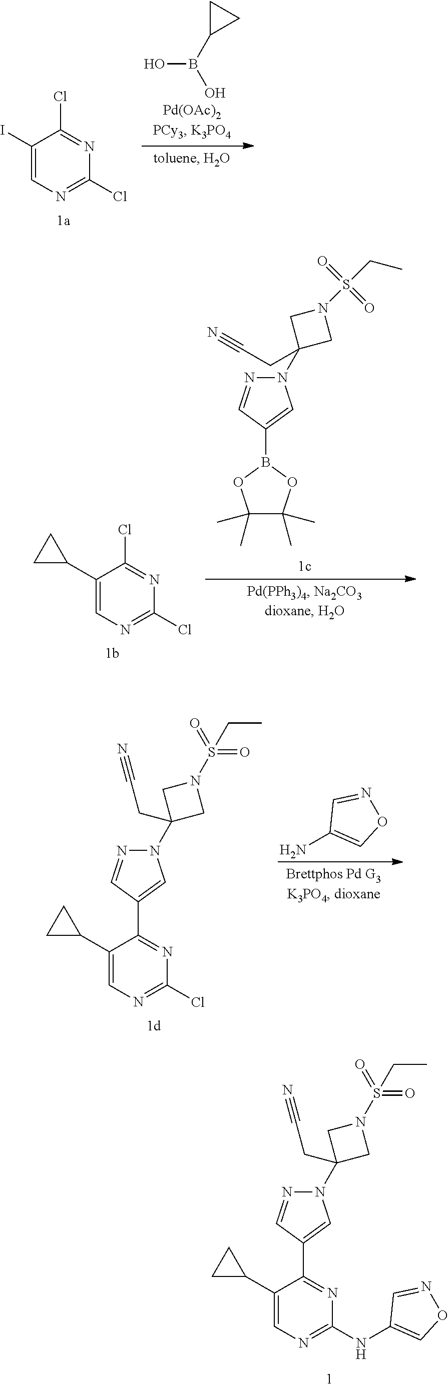

Step 1: A mixture of 1a (9.2 g, 33.5 mmol, 1.0 eq), cyclopropylboronic acid (3.7 g, 43.5 mmol, 1.3 eq), Pd(OAc)₂ (1.5 g, 6.7 mmol, 0.2 eq), PCy₃ (3.7 g, 13.4 mmol, 0.4 eq) and K₃PO₄ (56.7 g, 267.6 mmol, 8.0 eq) in toluene (460 mL)/H₂O (92 mL) was stirred at 105° C. overnight, then cooled to room temperature. The resulting mixture was concentrated and the residue was purified on silica gel column flash chromatography (PE/EA=10/1) to afford 1b (700 mg, 11.0% yield) as yellow oil. LC/MS: [M+H]⁺:189.2.

Step 2: A mixture of 1b (388 mg, 2.06 mmol, 2.0 eq), 2-(1-(ethylsulfonyl)-3-(4-(4,4,5,5-tetramethyl-1,3,2-dioxaborolan-2-yl)-1H-pyrazol-1-yl)azetidin-3-yl)acetonitrile 1c (390 mg, 1.03 mmol, 1.0 eq), Pd(PPh₃)₄ (115 mg, 0.10 mmol, 0.05 eq), and Na₂CO₃ (164 mg, 1.55 mmol, 1.5 eq) in dioxane (6 mL)/H₂O (1 mL) was stirred at 80° C. overnight, then cooled to room temperature. The resulting mixture was concentrated and the residue purified on silica gel column flash chromatography (PE/EA=1/1) to afford 1d (300 mg, 71.8% yield) as a yellow solid. LC/MS: [M+H]⁺:407.1.

Step 3: A mixture of 1d (100 mg, 0.25 mmol, 1.0 eq), isoxazol-4-amine (31 mg, 0.37 mmol, 1.5 eq), Brettphos Pd G₃ (23 mg, 0.025 mmol, 0.1 eq) and K₃PO₄ (104 mg, 0.50 mmol, 2.0 eq) in dioxane (5 mL) was stirred at 100° C. overnight under N₂. The resulting mixture was concentrated, purified by column chromatography on silica gel (DCM/MeOH=20/1) to afford crude product (50 mg), and then the crude product was purified by Prep-HPLC (0.1% NH₃H₂O) to afford 1 (9.60 mg, 0.021 mmol, 8.5% yield) as a white solid. Rt: 1.525 min. LCMS: [M+H]⁺: 455.1. ¹H NMR (400 MHz, DMSO-d₆): δ 9.58 (s, 1H), 9.13 (s, 1H), 8.76 (s, 1H), 8.61 (s, 1H), 8.40 (s, 1H), 8.32 (s, 1H), 4.56 (d, J=9.2 Hz, 2H), 4.25 (d, J=9.2 Hz, 2H), 3.69 (s, 2H), 3.27-3.21 (m, 2H), 2.03-1.99 (m, 1H), 1.25 (t, J=7.6 Hz, 3H), 1.05-1.00 (m, 2H), 0.64-0.61 (m, 2H).

### Example 2: Preparation of 2-(3-(4-(5-cyclopropyl-2-((1-methyl-1H-pyrazol-4-yl) amino) pyrimidin-4-yl)-1H-pyrazol-1-yl)-1-(ethylsulfonyl) azetidin-3-yl) acetonitrile (2)

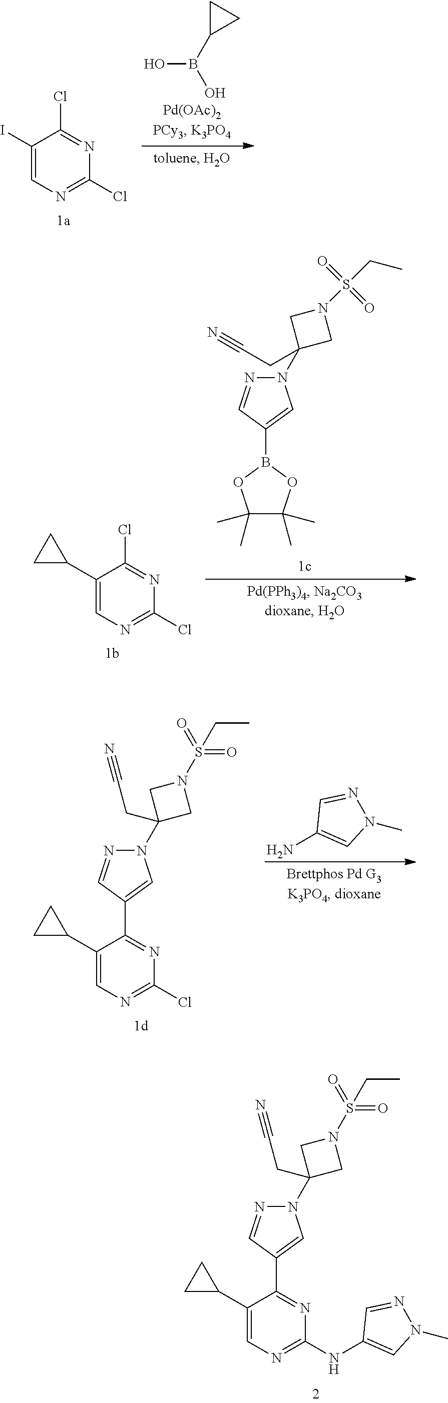

Step 1: A mixture of 1a (9.2 g, 33.5 mmol, 1.0 eq), cyclopropylboronic acid (3.7 g, 43.5 mmol, 1.3 eq), Pd(OAc)₂ (1.5 g, 6.7 mmol, 0.2 eq), Pcy₃ (3.7 g, 13.4 mmol, 0.4 eq) and K₃PO₄ (56.7 g, 267.6 mmol, 8.0 eq) in toluene (460 mL)/H₂O (92 mL) was stirred at 105° C. overnight, then cooled to room temperature. The resulting mixture was concentrated and the residue was purified on silica gel column flash chromatography (PE/EA=10/1) to afford 1b (700 mg, 11.0% yield) as yellow oil. LC/MS: [M+H]⁺:189.2.

Step 2: A mixture of 1b (388 mg, 2.06 mmol, 2.0 eq), 2-(1-(ethylsulfonyl)-3-(4-(4,4,5,5-tetramethyl-1,3,2-dioxaborolan-2-yl)-1H-pyrazol-1-yl)azetidin-3-yl)acetonitrile 1c (390 mg, 1.03 mmol, 1.0 eq), Pd(PPh₃)₄ (115 mg, 0.10 mmol, 0.05 eq), and Na₂CO₃ (164 mg, 1.55 mmol, 1.5 eq) in dioxane (6 mL)/H₂O (1 mL) was stirred at 80° C. overnight, then cooled to room temperature.

The resulting mixture was concentrated and the residue purified on silica gel column flash chromatography (PE/EA=1/1) to afford 1d (300 mg, 71.8% yield) as a yellow solid. LC/MS: [M+H]⁺:407.1.

Step 3: A mixture of 1d (100 mg, 0.25 mmol, 1.0 eq), 1-methyl-1H-pyrazol-4-amine (36 mg, 0.37 mmol, 1.5 eq), Brettphos Pd G₃ (23 mg, 0.025 mmol, 0.1 eq) and K₃PO₄ (104 mg, 0.50 mmol, 2.0 eq) in dioxane (5 mL) was stirred at 100° C. overnight under N₂. The resulting mixture was concentrated, purified by column chromatography on silica gel (DCM/MeOH=20/1) to afford crude product (120 mg), and then the crude product was purified by Prep-HPLC (0.1% NH₃H₂O) to afford 2 (19.8 mg, 0.042 mmol, 17.2% yield) as a white solid. Rt: 1.348 min. LCMS: [M+H]⁺: 468.3. ¹H NMR (400 MHz, DMSO-d₆): δ 9.26 (s, 1H), 8.73 (s, 1H), 8.37 (s, 1H), 8.23 (s, 1H), 7.92 (s, 1H), 7.47 (s, 1H), 4.55 (d, J=9.2 Hz, 2H), 4.24 (d, J=9.6 Hz, 2H), 3.82 (s, 3H), 3.69 (s, 2H), 3.27-3.21 (m, 2H), 2.00-1.96 (m, 1H), 1.25 (t, J=7.2 Hz, 3H), 1.03-0.98 (m, 2H), 0.61-0.57 (m, 2H).

### Example 3: Preparation of 2-(1-(ethylsulfonyl)-3-(4-(2-(isoxazol-4-ylamino)-5-methylpyrimidin-4-yl)-1H-pyrazol-1-yl) azetidin-3-yl) acetonitrile (3)

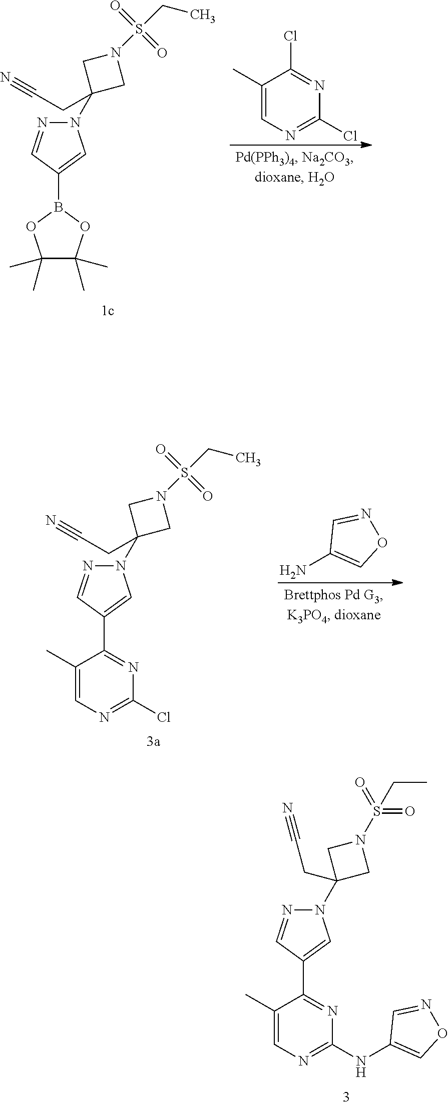

Step 1: A mixture of 1c (500 mg, 1.32 mmol, 1.0 eq), 2,4-dichloro-5-methylpyrimidine (643 mg, 3.96 mmol, 3.0 eq), Pd(PPh₃)₄ (150 mg, 0.13 mmol, 0.1 eq), and Na₂CO₃ (280 mg, 2.64 mmol, 2.0 eq) in dioxane (10 mL) and H₂O (1 mL) was stirred at 80° C. for 16 h, then cooled to room temperature. The resulting mixture was concentrated and the residue was purified on silica gel column flash chromatography (PE/EA=1/1) to afford 3a (505 mg, crude) as a white solid. LC/MS: [M+H]⁺:381.0.

Step 2: A mixture of 3a (100 mg, 0.26 mmol, 1.0 eq), isoxazol-4-amine (44 mg, 0.52 mmol, 2.0 eq), Brettphos Pd G₃ (24 mg, 0.026 mmol, 0.1 eq) and K₃PO₄ (138 mg, 0.65 mmol, 2.5 eq) in dioxane (5 mL) was stirred at 100° C. overnight under N₂. The resulting mixture was concentrated, purified by column chromatography on silica gel (DCM/MeOH=20/1) to afford crude product, and then the crude product was purified by Prep-HPLC (0.1% NH₃H₂O) to afford 3 (9.23 mg, 0.022 mmol, 8.2% yield) as a white solid. Rt: 1.168 min. LCMS: [M+H]⁺: 429.2. ¹H NMR (400 MHz, DMSO-d₆): δ 9.53 (s, 1H), 9.13 (s, 1H), 8.66 (s, 1H), 8.60 (s, 1H), 8.37 (s, 1H), 8.28 (s, 1H), 4.57 (d, J=9.2 Hz, 2H), 4.25 (d, J=9.2 Hz, 2H), 3.67 (s, 2H), 3.26-3.23 (m, 2H), 2.35 (s, 3H), 1.26-1.23 (m, 3H).

### Example 4: Preparation of (R)-3-cyclopentyl-3-(4-(5-methyl-2-((1-methyl-1H-pyrazol-4-yl) amino) pyrimidin-4-yl)-1H-pyrazol-1-yl) propanenitrile (4)

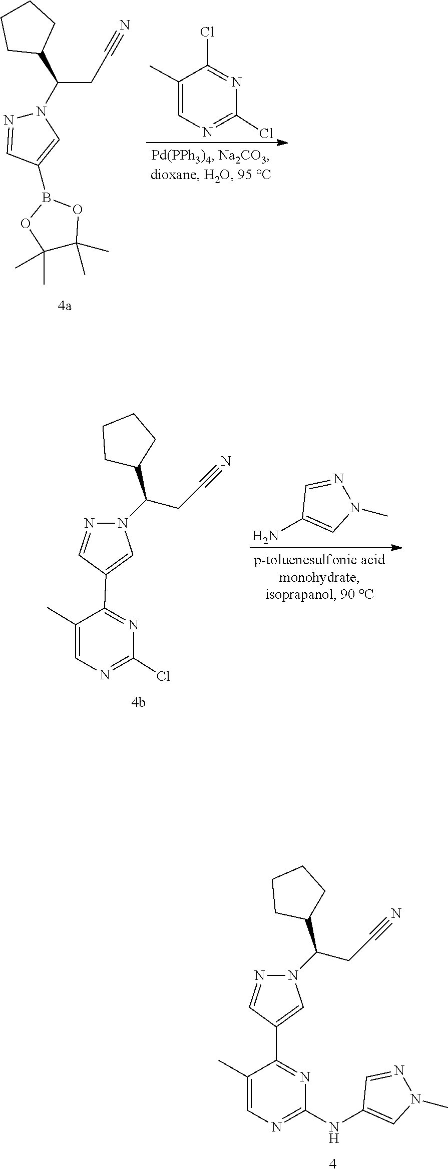

Step 1: Compound 4a (15.2 g, 48.2 mmol, 1.0 eq), 2, 4-dichloro-5-methylpyrimidine (23.6 g, 145 mmol, 3.0 eq), and anhydrous Na₂CO₃ (15.4 g, 145 mmol, 2.0 eq) were added to a mixture solvent of dioxane (400 mL) and H₂O (40 mL) at room temperature. Pd(PPh₃)₄ (5.57 g, 4.82 mmol) was then added quickly and purged with nitrogen three times. The reaction mixture was heated at 95° C. for 18 hours under N₂ atmosphere. Upon the reaction completed, the reaction mixture was filtered, and the filtrate was concentrated to obtain a crude product. The residue was purified by column chromatography on silica gel (PE/EA=3/1) to afford compound 4b (18 g) as an oil. Rt: 1.820 min. LCMS: [M+H]⁺: 316.0.

Step 2: Compound 4b (16.8 g, 53.2 mmol, 1.0 eq), 1-methyl-1H-pyrazol-4-amine (25.8 g, 266 mmol), and p-toluenesulfonic acid monohydrate (30.3 g, 160 mmol) were added to isopropanol (1000 mL), and the mixture was stirred at 90° C. for 18 hours under N₂ atmosphere. Upon the reaction completed, the reaction mixture was concentrated and purified by column chromatography on silica gel (DCM/MeOH=10/1) to afford a crude product. The crude product was further purified by Prep-HPLC to afford compound 4 (12 g, 60% yield) as a white solid. Rt: 1.569 min. LCMS: [M+H]⁺: 377.2. ¹H NMR (400 MHz, DMSO) δ 9.17 (s, 1H), 8.48 (s, 1H), 8.24 (s, 1H), 8.17 (s, 1H), 7.90 (s, 1H), 7.48 (s, 1H), 4.59-4.54 (m, 1H), 3.82 (s, 3H), 3.27-3.15 (m, 2H), 2.45-2.38 (m, 1H), 2.29 (s, 3H), 1.84-1.80 (m, 1H), 1.62-1.44 (m, 4H), 1.35-1.16 (m, 3H).

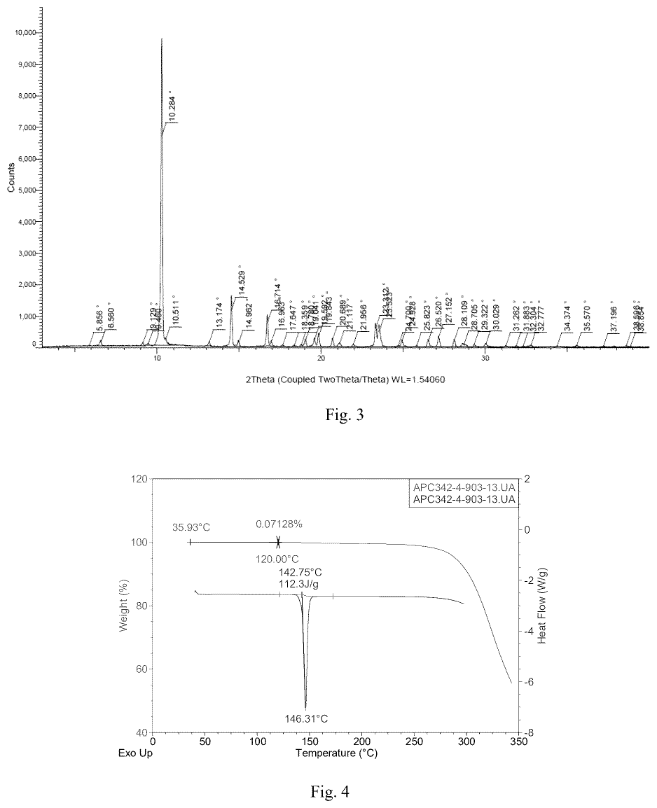

The solid was confirmed to be free base Form A of the present disclosure. The XRPD pattern is substantially as depicted in FIG. 3, and the XRPD data are listed in Table 3. The TGA curve of Form A in the present disclosure shows about 0.07% weight loss when heated to 120° C., which is substantially as depicted in FIG. 4. The DSC curve of Form A in the present disclosure is also depicted in FIG. 4, which shows one endothermic peak at around 143° C. corresponding to the melting endothermic peak. Without any limitation being implied, Form A is an anhydrate.

TABLE 3
XRPD peak list

| | | | Net | Gross | Rel. | |
|---|---|---|---|---|---|---|
| | Angle | d Value | Inten- | Inten- | Inten- | Inten- |
| Index | (°) | (Å) | sity | sity | sity | sity |
| 1 | 5.856 | 15.07997 | 13.5163 | 65.6638 | 0.2% | 65.6638 |
| 2 | 6.560 | 13.46247 | 90.7108 | 152.3862 | 1.4% | 152.386 |
| 3 | 9.129 | 9.67888 | 76.6933 | 144.4753 | 1.2% | 144.475 |
| 4 | 9.460 | 9.34123 | 53.6517 | 123.2406 | 0.8% | 123.241 |
| 5 | 10.284 | 8.59465 | 6643.1350 | 6720.4190 | 100.0% | 6720.42 |
| 6 | 10.511 | 8.40972 | 158.7177 | 237.3816 | 2.4% | 237.382 |
| 7 | 13.174 | 6.71507 | 109.5005 | 160.3384 | 1.6% | 160.338 |
| 8 | 14.529 | 6.09172 | 1135.0330 | 1182.8310 | 17.1% | 1182.83 |
| 9 | 14.962 | 5.91655 | 93.2598 | 138.9585 | 1.4% | 138.959 |
| 10 | 16.714 | 5.30012 | 721.5863 | 771.1036 | 10.9% | 771.104 |
| 11 | 16.963 | 5.22264 | 130.8669 | 180.1233 | 2.0% | 180.123 |
| 12 | 17.647 | 5.02184 | 26.4311 | 70.7408 | 0.4% | 70.7408 |
| 13 | 18.359 | 4.82873 | 27.2364 | 70.9215 | 0.4% | 70.9215 |
| 14 | 18.780 | 4.72140 | 28.9468 | 74.7392 | 0.4% | 74.7392 |
| 15 | 19.041 | 4.65717 | 176.3729 | 223.5949 | 2.7% | 223.595 |
| 16 | 19.592 | 4.52741 | 201.6987 | 248.6722 | 3.0% | 248.672 |
| 17 | 19.843 | 4.47063 | 303.5757 | 348.7130 | 4.6% | 348.713 |
| 18 | 20.689 | 4.28973 | 209.8084 | 251.8533 | 3.2% | 251.853 |
| 19 | 21.117 | 4.20376 | 80.4620 | 122.1204 | 1.2% | 122.12 |
| 20 | 21.956 | 4.04501 | 55.5562 | 93.1114 | 0.8% | 93.1114 |
| 21 | 23.312 | 3.81266 | 572.7300 | 614.3822 | 8.6% | 614.382 |
| 22 | 23.523 | 3.77898 | 455.0738 | 497.3672 | 6.9% | 497.367 |
| 23 | 24.700 | 3.60154 | 29.4235 | 64.0738 | 0.4% | 64.0738 |
| 24 | 24.928 | 3.56903 | 180.3056 | 215.5691 | 2.7% | 215.569 |
| 25 | 25.823 | 3.44741 | 45.1470 | 80.2097 | 0.7% | 80.2097 |
| 26 | 26.520 | 3.35833 | 158.6529 | 199.7945 | 2.4% | 199.795 |
| 27 | 27.152 | 3.28160 | 283.3949 | 324.7568 | 4.3% | 324.757 |
| 28 | 28.109 | 3.17200 | 182.3637 | 219.1447 | 2.7% | 219.145 |
| 29 | 28.705 | 3.10746 | 68.9722 | 104.3394 | 1.0% | 104.339 |
| 30 | 29.322 | 3.04342 | 68.1542 | 99.1213 | 1.0% | 99.1213 |
| 31 | 30.029 | 2.97339 | 79.1439 | 109.6487 | 1.2% | 109.649 |
| 32 | 31.262 | 2.85892 | 35.0451 | 65.4840 | 0.5% | 65.484 |
| 33 | 31.883 | 2.80462 | 26.7329 | 54.5220 | 0.4% | 54.522 |
| 34 | 32.304 | 2.76905 | 17.8404 | 44.1906 | 0.3% | 44.1906 |
| 35 | 32.777 | 2.73015 | 35.7948 | 64.5379 | 0.5% | 64.5379 |
| 36 | 34.374 | 2.60683 | 23.9690 | 56.4113 | 0.4% | 56.4113 |
| 37 | 35.570 | 2.52191 | 40.0003 | 66.0410 | 0.6% | 66.041 |
| 38 | 37.196 | 2.41531 | 12.3020 | 37.4652 | 0.2% | 37.4652 |
| 39 | 38.596 | 2.33084 | 42.3423 | 70.0044 | 0.6% | 70.0044 |
| 40 | 38.854 | 2.31597 | 15.5981 | 42.6651 | 0.2% | 42.6651 |

### Example 5: Preparation of (R)-3-cyclopentyl-3-(4-(2-(isoxazol-4-ylamino)-5-methylpyrimidin-4-yl)-1H-pyrazol-1-yl) propanenitrile (5)

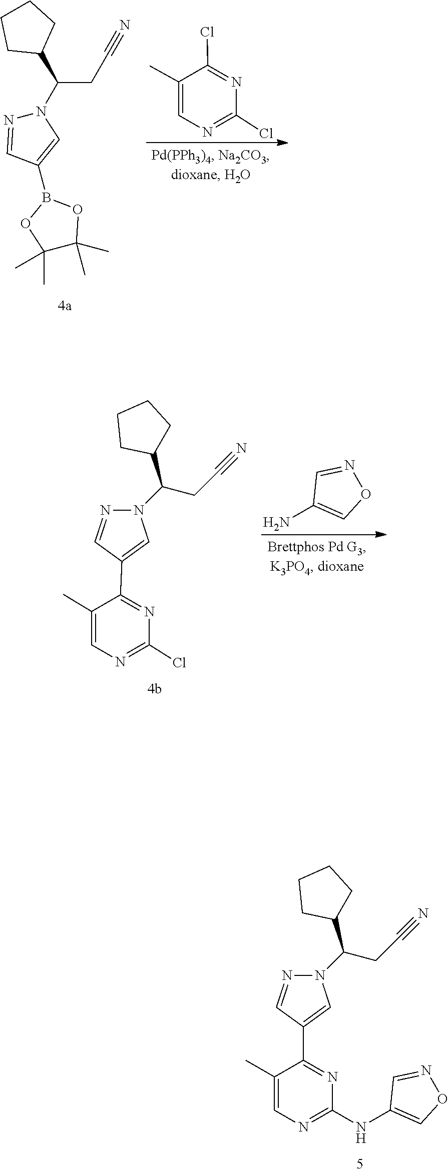

Step 1: A mixture of 4a (500 mg, 1.59 mmol, 1.0 eq) and 2, 4-dichloro-5-methylpyrimidine (778 mg, 4.77 mmol, 3.0 eq) in dioxane (20 mL) and H₂O (2 mL) was added Na₂CO₃ (337 mg, 3.18 mmol, 2.0 eq) and Pd(PPh₃)₄ (185 mg, 0.16 mmol, 0.1 eq). After addition, the mixture was degassed under N₂ for three times and stirred at 80° C. under N₂ atmosphere for 16 hours. The reaction mixture was concentrated. The residue was purified by column chromatography on silica gel (PE/EA=4/1) to give to afford 4b (771 mg, crude) as a brown solid. Rt: 1.575 min. LCMS: [M+H]⁺: 316.2.

Step 2: A mixture of 4b (450 mg, 1.43 mmol, 1.0 eq), isoxazol-4-amine (239 mg, 2.86 mmol, 2.0 eq), Brettphos Pd G₃ (65 mg, 0.072 mmol, 0.05 eq) and K₃PO₄ (758 mg, 3.58 mmol, 2.5 eq) in dioxane (20 mL) was stirred at 100° C. under N₂ atmosphere for 16 hours. The resulting mixture was concentrated, purified by column chromatography on silica gel (DCM/MeOH=10/1) to afford crude product (110 mg), and then the crude product was purified by Prep-HPLC (0.1% NH₃H₂O) to afford 5 (36.6 mg, 0.10 mmol, 7.1% yield) as a white solid. Rt: 1.891 min. LCMS: [M+H]⁺: 364.2. ¹H NMR (400 MHz, DMSO) δ 9.49 (s, 1H), 9.13 (s, 1H), 8.60 (s, 1H), 8.56 (s, 1H), 8.33 (s, 1H), 8.18 (s, 1H), 4.61-4.56 (m, 1H), 3.27-3.16 (m, 2H), 2.49-2.39 (m, 1H), 2.33 (s, 3H), 1.84-1.80 (m, 1H), 1.63-1.43 (m, 4H), 1.35-1.18 (m, 3H).

### Example 6: Preparation of (R)-3-cyclopentyl-3-(4-(5-methyl-2-((1-((tetrahydro-2H-pyran-4-yl) methyl)-1H-pyrazol-4-yl) amino) pyrimidin-4-yl)-1H-pyrazol-1-yl)propanenitrile (6)

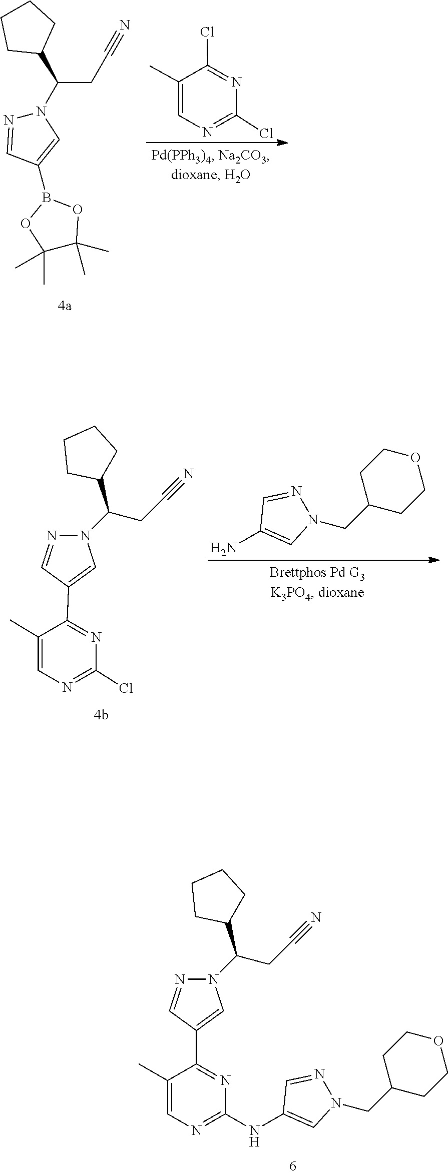

Step 1: A mixture of 4a (500 mg, 1.59 mmol, 1.0 eq) and 2, 4-dichloro-5-methylpyrimidine (778 mg, 4.77 mmol, 3.0 eq) in dioxane (20 mL) and H₂O (2 mL) was added Na₂CO₃ (337 mg, 3.18 mmol, 2.0 eq) and Pd(PPh₃)₄ (185 mg, 0.16 mmol, 0.1 eq). After addition, the mixture was degassed under N₂ for three times and stirred at 80° C. under N₂ atmosphere for 16 hours. The reaction mixture was concentrated. The residue was purified by column chromatography on silica gel (PE/EA=4/1) to give to afford 4b (771 mg, crude) as a brown solid. Rt: 1.575 min. LCMS: [M+H]⁺: 316.2.

Step 2: A mixture of 4b (130 mg, 0.41 mmol, 1.0 eq), 1-((tetrahydro-2H-pyran-4-yl)methyl)-1H-pyrazol-4-amine (149 mg, 0.82 mmol, 2.0 eq), Brettphos Pd G₃ (36 mg, 0.041 mmol, 0.1 eq) and K₃PO₄ (218 mg, 1.03 mmol, 2.5 eq) in dioxane (10 mL) was stirred at 120° C. under N₂ atmosphere for 16 hours. The resulting mixture was filtered, and the residue was concentrated, purified by Prep-HPLC (0.1% NH₃H₂O) to afford 6 (14.72 mg, 0.032 mmol, 7.7% yield) as a white solid. Rt: 1.514 min. LCMS: [M+H]⁺: 461.3. ¹H NMR (400 MHz, DMSO) δ 9.18 (s, 1H), 8.48 (s, 1H), 8.24 (s, 1H), 8.16 (s, 1H), 7.93 (s, 1H), 7.50 (s, 1H), 4.57-4.56 (m, 1H), 3.99-3.97 (m, 2H), 3.84-3.80 (m, 2H), 3.27-3.19 (m, 4H), 2.45-2.38 (m, 1H), 2.29 (s, 3H), 2.06-1.98 (m, 1H), 1.85-1.78 (m, 1H), 1.65-1.39 (m, 6H), 1.35-1.13 (m, 5H).

### Example 7: Preparation of (R)-3-cyclopentyl-3-(4-(5-methyl-2-((1-(tetrahydro-2H-pyran-4-yl)-1H-pyrazol-4-yl) amino) pyrimidin-4-yl)-1H-pyrazol-1-yl)propanenitrile (7)

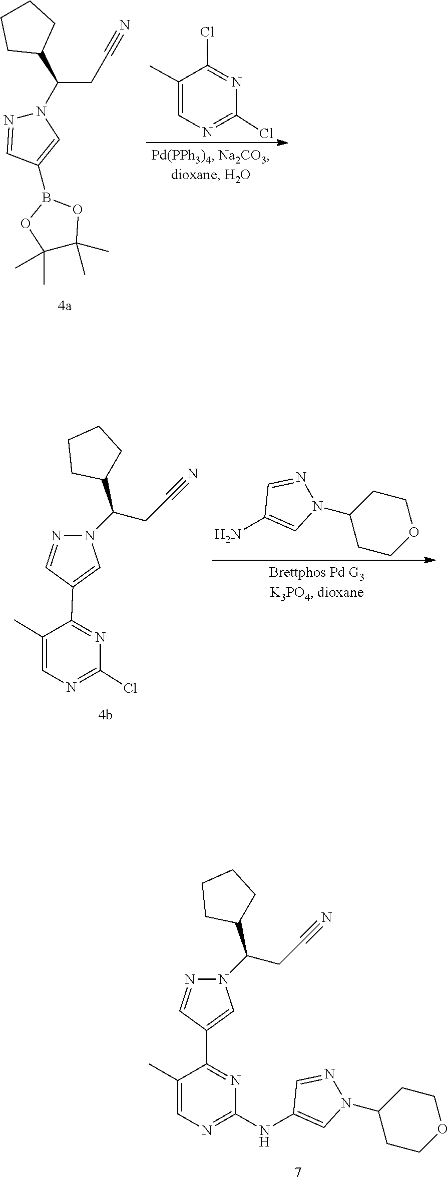

Step 1: A mixture of 4a (500 mg, 1.59 mmol, 1.0 eq) and 2, 4-dichloro-5-methylpyrimidine (778 mg, 4.77 mmol, 3.0 eq) in dioxane (20 mL) and H₂O (2 mL) was added Na₂CO₃ (337 mg, 3.18 mmol, 2.0 eq) and Pd(PPh₃)₄ (185 mg, 0.16 mmol, 0.1 eq). After addition, the mixture was degassed under N₂ for three times and stirred at 80° C. under N₂ atmosphere for 16 hours. The reaction mixture was concentrated. The residue was purified by column chromatography on silica gel (PE/EA=4/1) to give to afford 4b (771 mg, crude) as a brown solid. Rt: 1.575 min. LCMS: [M+H]⁺: 316.2.

Step 2: A mixture of 4b (130 mg, 0.41 mmol, 1.0 eq), 1-(tetrahydro-2H-pyran-4-yl)-1H-pyrazol-4-amine (138 mg, 0.82 mmol, 2.0 eq), Brettphos Pd G₃ (36 mg, 0.041 mmol, 0.1 eq) and K₃PO₄ (218 mg, 1.03 mmol, 2.5 eq) in dioxane (10 mL) was stirred at 120° C. under N₂ atmosphere for 16 hours. The resulting mixture was filtered, and the residue was concentrated, purified by Prep-HPLC (0.1% NH₃H₂O) to afford 7 (21.47 mg, 0.048 mmol, 11.6% yield) as a white solid. Rt: 1.484 min. LCMS: [M+H]⁺: 447.2. ¹H NMR (400 MHz, DMSO) δ 9.17 (s, 1H), 8.48 (s, 1H), 8.24 (s, 1H), 8.15 (s, 1H), 7.98 (s, 1H), 7.54 (s, 1H), 4.57-4.56 (m, 1H), 4.39-4.36 (m, 1H), 3.98-3.95 (m, 2H), 3.50-3.43 (m, 2H), 3.29-3.19 (m, 2H), 2.44-2.38 (m, 1H), 2.29 (s, 3H), 1.96-1.81 (m, 5H), 1.62-1.32 (m, 4H), 1.30-1.18 (m, 3H).

### Example 8: Preparation of (R)-3-cyclopentyl-3-(4-(5-methyl-2-((1-(oxetan-3-yl)-1H-pyrazol-4-yl) amino) pyrimidin-4-yl)-1H-pyrazol-1-yl) propanenitrile (8)

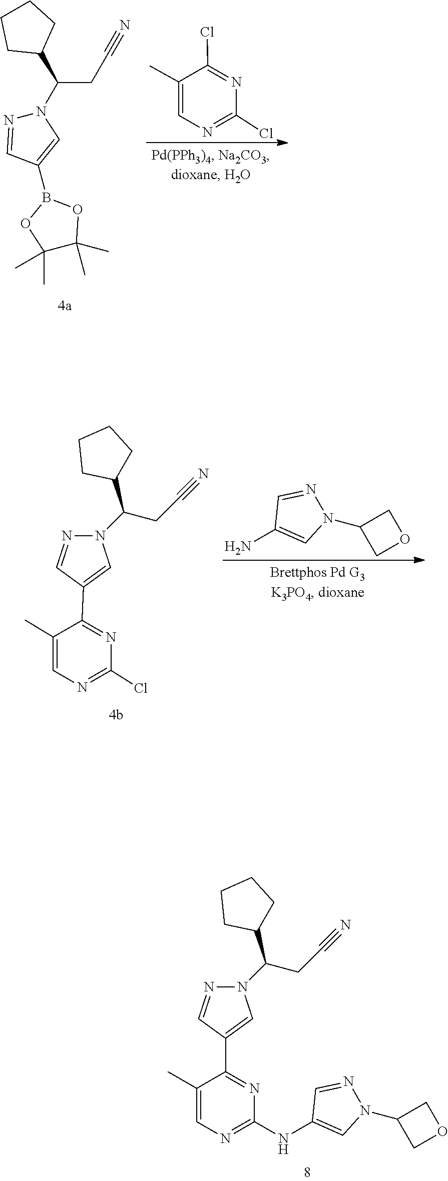

Step 1: A mixture of 4a (500 mg, 1.59 mmol, 1.0 eq) and 2, 4-dichloro-5-methylpyrimidine (778 mg, 4.77 mmol, 3.0 eq) in dioxane (20 mL) and H₂O (2 mL) was added Na₂CO₃ (337 mg, 3.18 mmol, 2.0 eq) and Pd(PPh₃)₄ (185 mg, 0.16 mmol, 0.1 eq). After addition, the mixture was degassed under N₂ for three times and stirred at 80° C. under N₂ atmosphere for 16 hours. The reaction mixture was concentrated. The residue was purified by column chromatography on silica gel (PE/EA=4/1) to give to afford 4b (771 mg, crude) as a brown solid. Rt: 1.575 min. LCMS: [M+H]⁺: 316.2.

Step 2: A mixture of 4b (130 mg, 0.41 mmol, 1.0 eq), 1-(oxetan-3-yl)-1H-pyrazol-4-amine (115 mg, 0.82 mmol, 2.0 eq), Brettphos Pd G₃ (36 mg, 0.041 mmol, 0.1 eq) and K₃PO₄ (218 mg, 1.03 mmol, 2.5 eq) in dioxane (10 mL) was stirred at 120° C. under N₂ atmosphere for 16 hours. The resulting mixture was filtered, and the residue was concentrated, purified by Prep-HPLC (0.1% NH₃H₂O) to afford 8 (47.39 mg, 0.113 mmol, 27.4% yield) as a white solid. Rt: 1.433 min. LCMS: [M+H]⁺: 419.2. ¹H NMR (400 MHz, DMSO) δ 9.27 (s, 1H), 8.49 (s, 1H), 8.25 (s, 1H), 8.18 (s, 1H), 8.08 (s, 1H), 7.66 (s, 1H), 5.62-5.55 (m, 1H), 4.93-4.87 (m, 4H), 4.59-4.53 (m, 1H), 3.27-3.19 (m, 2H), 2.45-2.41 (m, 1H), 2.30 (s, 3H), 1.86-1.78 (m, 1H), 1.61-1.44 (m, 4H), 1.34-1.18 (m, 3H).

### Example 9: Preparation of (R)-3-cyclopentyl-3-(4-(5-methyl-2-((1-(oxetan-3-ylmethyl)-1H-pyrazol-4-yl) amino) pyrimidin-4-yl)-1H-pyrazol-1-yl) propanenitrile (9)

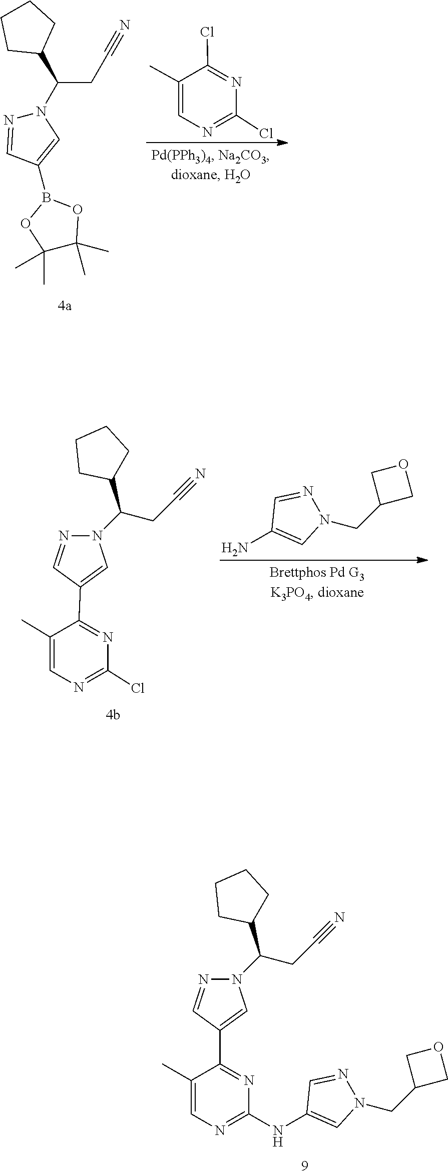

Step 1: A mixture of 4a (500 mg, 1.59 mmol, 1.0 eq) and 2, 4-dichloro-5-methylpyrimidine (778 mg, 4.77 mmol, 3.0 eq) in dioxane (20 mL) and H₂O (2 mL) was added Na₂CO₃ (337 mg, 3.18 mmol, 2.0 eq) and Pd(PPh₃)₄ (185 mg, 0.16 mmol, 0.1 eq). After addition, the mixture was degassed under N₂ for three times and stirred at 80° C. under N₂ atmosphere for 16 hours. The reaction mixture was concentrated. The residue was purified by column chromatography on silica gel (PE/EA=4/1) to give to afford 4b (771 mg, crude) as a brown solid. Rt: 1.575 min. LCMS: [M+H]⁺: 316.2.

Step 2: A mixture of 4b (130 mg, 0.41 mmol, 1.0 eq), 1-(oxetan-3-ylmethyl)-1H-pyrazol-4-amine (125 mg, 0.82 mmol, 2.0 eq), Brettphos Pd G₃ (36 mg, 0.041 mmol, 0.1 eq) and K₃PO₄ (218 mg, 1.03 mmol, 2.5 eq) in dioxane (10 mL) was stirred at 120° C. under N₂ atmosphere for 16 hours. The resulting mixture was filtered, and the residue was concentrated, purified by Prep-HPLC (0.1% NH₃H₂O) to afford 9 (29.0 mg, 0.066 mmol, 16.2% yield) as a white solid. Rt: 1.391 min. LCMS: [M+H]⁺: 433.2. ¹H NMR (400 MHz, DMSO) δ 9.18 (s, 1H), 8.48 (s, 1H), 8.24 (s, 1H), 8.17 (s, 1H), 7.95 (s, 1H), 7.50 (s, 1H), 4.65-4.62 (m, 2H), 4.58-4.55 (m, 1H), 4.44-4.39 (m, 4H), 3.42-3.38 (m, 1H), 3.22-3.18 (m, 2H), 2.42-2.40 (m, 1H), 2.20 (s, 3H), 1.83-1.81 (m, 1H), 1.61-1.44 (m, 4H), 1.34-1.19 (m, 3H).

### Example 10: Preparation of (R)-3-(4-(5-chloro-2-((1-(2-hydroxyethyl)-1H-pyrazol-4-yl)amino)pyrimidin-4-yl)-1H-pyrazol-1-yl)-3-cyclopentylpropanenitrile (10)

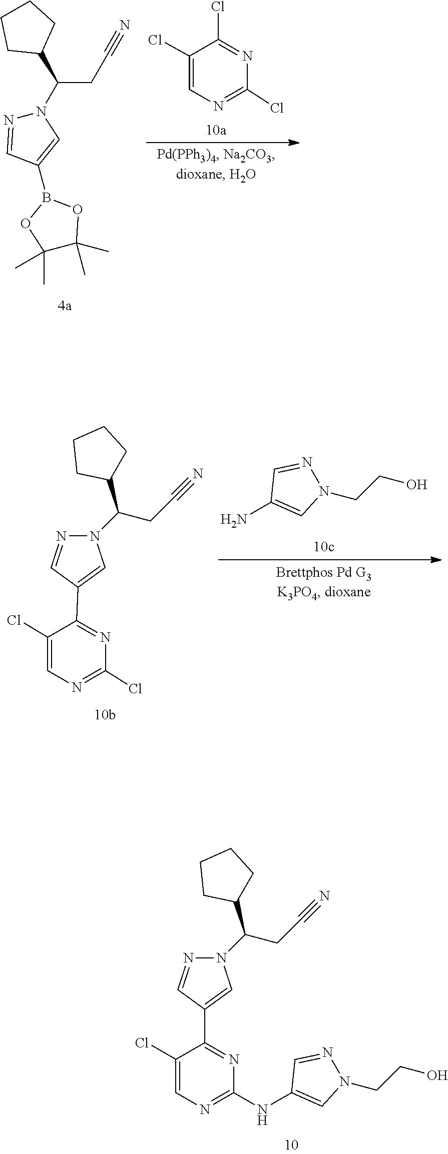

Step 1: A mixture of 4a (600 mg, 1.90 mmol), 10a (1.06 g, 5.80 mmol), Pd(PPh₃)₄ (100 mg) and Na₂CO₃ (615 mg, 5.80 mmol) in dioxane (50 mL) and H₂O (5 ml) was stirred at 80° C. overnight under N₂. The resulting mixture was filtered, and the residue was concentrated, purified on silica gel column flash chromatography (PE/EA=1/1) to afford 10b (600 mg, 94% yield) as brown solid. LC/MS: [M+H]⁺:336.

Step 2: A mixture of 10b (410 mg, 1.23 mmol), 10c (280 mg, 2.21 mmol), Brettphos Pd G₃ (111 mg, 0.123 mmol) and K₃PO₄ (391 mg, 1.85 mmol) in dioxane (20 mL) was stirred at 100° C. overnight under N₂. The resulting mixture was filtered, and the residue was concentrated, purified by Prep-HPLC to afford 10 (9.35 mg, 2% yield) as a yellow solid. LC/MS: [M+H]⁺:427.2. ¹H NMR (400 MHz, DMSO) δ 9.60 (s, 1H), 8.79-8.63 (m, 1H), 8.44 (s, 1H), 8.34 (s, 1H), 7.99 (s, 1H), 7.52 (s, 1H), 4.94 (s, 1H), 4.61-4.63 (m, 1H), 4.14 (s, 2H), 3.73 (dd, J=10.8, 5.6 Hz, 2H), 3.28-3.13 (m, 2H), 2.50 (dd, J=3.2 Hz, 1.6 Hz, 1H), 1.79-1.84 (m, 1H), 1.42-1.69 (m, 4H), 1.11-1.38 (m, 3H).

### Example 11: Preparation of ((R)-3-(4-(5-chloro-2-((1-(tetrahydro-2H-pyran-4-yl)-1H-pyrazol-4-yl)amino)pyrimidin-4-yl)-1H-pyrazol-1-yl)-3-cyclopentylpropanenitrile (11)

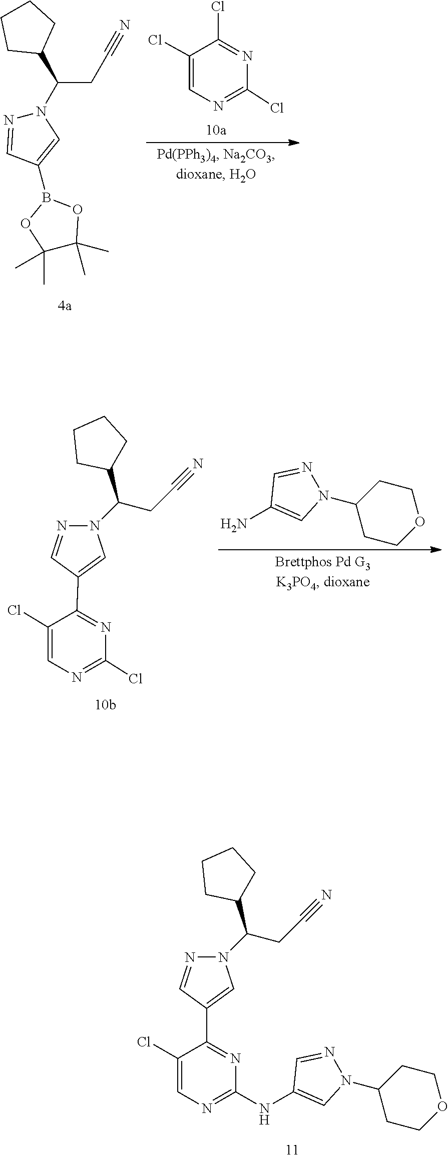

Step 1: A mixture of 4a (600 mg, 1.90 mmol), 10a (1.06 g, 5.80 mmol), Pd(PPh₃)₄ (100 mg) and Na₂CO₃ (615 mg, 5.80 mmol) in dioxane (50 mL) and H₂O (5 ml) was stirred at 80° C. overnight under N₂. The resulting mixture was filtered, and the residue was concentrated, purified on silica gel column flash chromatography (PE/EA=1/1) to afford 10b (600 mg, 94% yield) as brown solid. LC/MS: 336 [M+H]⁺.

Step 2: A mixture of 10b (200 mg, 0.59 mmol), 1-(tetrahydro-2H-pyran-4-yl)-1H-pyrazol-4-amine (199 mg, 1.19 mmol), Brettphos Pd G₃ (54 mg, 0.059 mmol) and K₃PO₄ (375 mg, 1.77 mmol) in dioxane (20 mL) was stirred at 100° C. overnight under N₂. The resulting mixture was filtered, and the residue was concentrated, purified by Prep-HPLC to afford 11 (14.38 mg, 5.2% yield) as a yellow solid. LC/MS: [M+H]⁺: 467.3. ¹H NMR (400 MHz, DMSO) δ 9.61 (s, 1H), 8.75 (s, 1H), 8.44 (s, 1H), 8.31 (s, 1H), 7.97 (s, 1H), 7.56 (s, 1H), 4.40-4.63 (m, 1H), 4.35-4.36 (m, 1H), 3.95-3.98 (m, 2H), 3.43-3.50 (m, 2H), 3.16-3.27 (m, 2H), 2.39-2.41 (m, 1H), 1.92-1.98 (m, 4H), 1.79-1.82 (m, 1H), 1.44-1.63 (m, 4H), 1.26-1.43 (m, 3H).

### Example 12: Preparation of 2-(1-(ethylsulfonyl)-3-(4-(2-((1-(2-hydroxyethyl)-1H-pyrazol-4-yl)amino)-5-methylpyrimidin-4-yl)-1H-pyrazol-1-yl)azetidin-3-yl)acetonitrile (12)

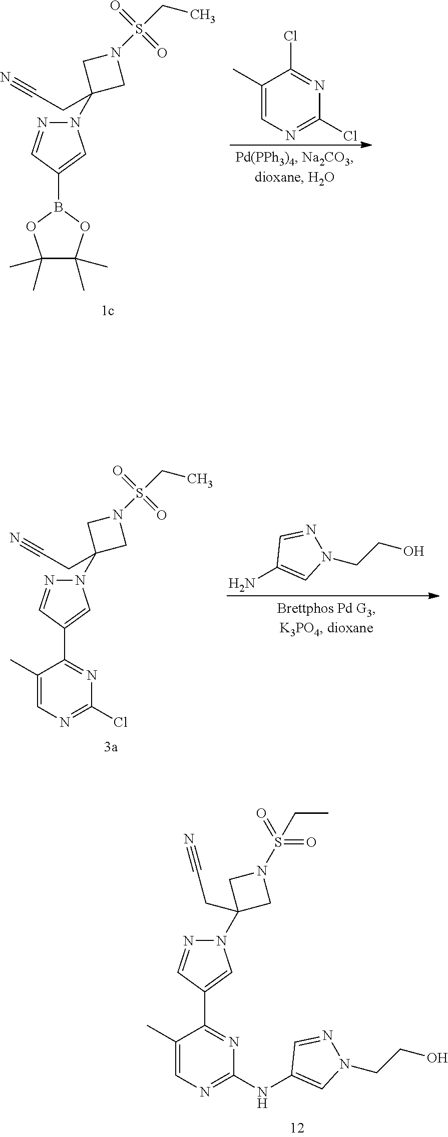

Step 1: A mixture of 1c (1 g, 2.63 mmol), 2, 4-dichloro-5-methylpyrimidine (1.28 g, 7.89 mmol), Pd(PPh₃)₄ (300 mg) and Na₂CO₃ (558 mg, 5.26 mmol) in dioxane (100 mL) and H₂O (10 ml) was stirred at 80° C. overnight under N₂. The resulting mixture was filtered, and the filtrate were extracted with EA (100 ml) three times, and the residue was concentrated, purified on silica gel column flash chromatography (DCM/MeOH=10/1) to afford 3a (1 g, 100% yield) as yellow solid. LC/MS: [M+H]⁺: 382.

Step 2: A mixture of 3a (300 mg, 0.789 mmol), 10c (180 mg, 1.42 mmol), Brettphos Pd G₃ (71 mg, 0.08 mmol) and K₃PO₄ (251 mg, 1.18 mmol) in dioxane (20 mL) was stirred at 100° C. overnight under N₂. The resulting mixture was filtered, and the residue was concentrated, purified by Prep-HPLC to afford 12 (62.90 mg, 17% yield) as a yellow solid. LC/MS: [M+H]⁺: 472.0. 1H NMR (400 MHz, DMSO) δ 9.21 (s, 1H), 8.60 (s, 1H), 8.27 (s, 2H), 8.00 (s, 1H), 7.49 (s, 1H), 4.90 (s, 1H), 4.56 (d, J=8.8 Hz, 2H), 4.24 (d, J=8.8 Hz, 2H), 4.12 (s, 2H), 3.72 (d, J=4.8 Hz, 2H), 3.66 (s, 2H), 3.26-3.19 (m, 2H), 2.32 (s, 3H), 1.24 (t, J=7.2 Hz, 3H).

### Example 13: Preparation of 2-(3-(4-(5-chloro-2-((1-(2-hydroxyethyl)-1H-pyrazol-4-yl)amino)pyrimidin-4-yl)-1H-pyrazol-1-yl)-1-(ethylsulfonyl)azetidin-3-yl)acetonitrile (13)

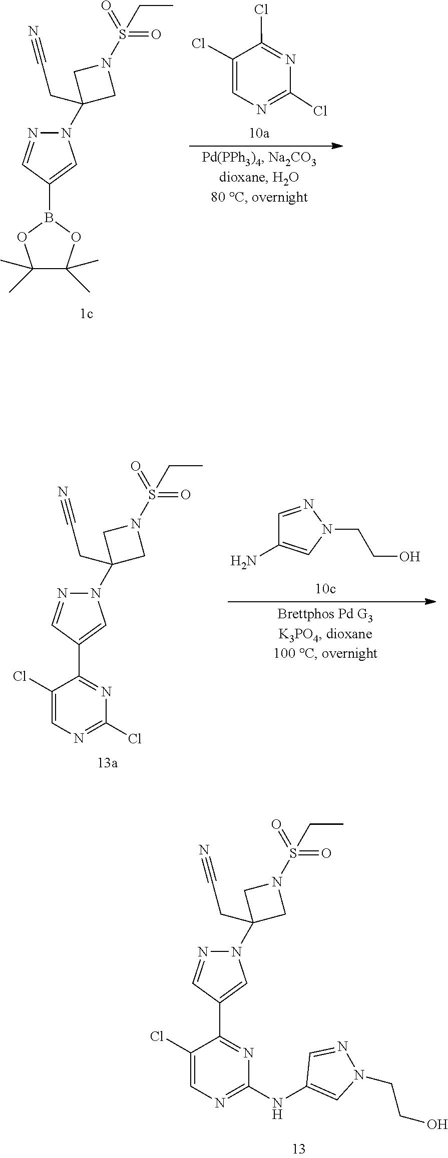 [SMILES: CCS(=O)(=O)N1CC(CC#N)(n2cc(B3OC(C)(C)C(C)(C)O3)cn2)C1 ; Clc1ncc(Cl)c(Cl)n1 ; CCS(=O)(=O)N1CC(CC#N)(n2cc(-c3nc(Cl)ncc3Cl)cn2)C1 ; Nc1cnn(CCO)c1 ; CCS(=O)(=O)N1CC(CC#N)(n2cc(-c3nc(Nc4cnn(CCO)c4)ncc3Cl)cn2)C1]

Step 1: A mixture of 1c (676 mg, 1.78 mmol), 10a (979 mg, 5.34 mmol), Pd(PPh₃)₄ (100 mg) and Na₂CO₃ (580 mg, 5.47 mmol) in dioxane (30 mL) and H₂O (3 ml) was stirred at 80° C. overnight under N₂. The resulting mixture was filtered, and the residue was concentrated, purified on silica gel column flash chromatography (PE/EA=1/1) to afford 13a (600 mg, 84% yield) as brown solid. LC/MS: [M+H]⁺: 401.

Step 2: A mixture of 13a (360 mg, 0.9 mmol), 10c (206 mg, 1.62 mmol), Brettphos Pd G₃ (81 mg, 0.09) and K₃PO₄ (286 mg, 1.35 mmol) in dioxane (10 mL) was stirred at 100° C. overnight under N₂. The resulting mixture was filtered, and the residue was concentrated, purified by Prep-HPLC to afford 13 (30 mg, 6% yield) as a yellow solid.

LC/MS: [M+H]⁺: 492.1. ¹H NMR (400 MHz, DMSO) δ 9.66 (s, 1H), 8.86 (s, 1H), 8.44 (d, J=28.4 Hz, 2H), 8.01 (s, 1H), 7.51 (s, 1H), 4.91 (s, 1H), 4.54 (d, J=12 Hz, 2H), 4.24 (d, J=9.2 Hz, 2H), 4.13 (d, J=5.6 Hz, 2H), 3.68-3.73 (m, 2H), 3.68 (s, 2H), 3.21-3.26 (m, 2H), 1.24 (t, J=8.0 Hz, 3H).

### Example 14: Preparation of 2-(1-(ethylsulfonyl)-3-(4-(5-methyl-2-((1-methyl-1H-pyrazol-3-yl)amino)pyrimidin-4-yl)-1H-pyrazol-1-yl)azetidin-3-yl)acetonitrile hydrochloride (14)

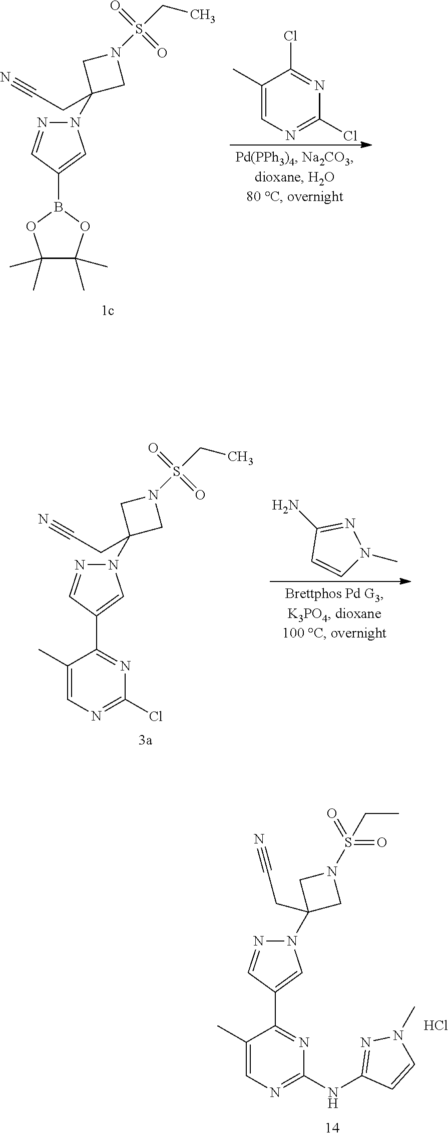

Step 1: A mixture of 1c (1 g, 2.63 mmol), 2, 4-dichloro-5-methylpyrimidine (1.28 g, 7.86 mmol), Pd(PPh₃)₄ (300 mg) and Na₂CO₃ (558 mg, 5.26 mmol) in dioxane (100 mL) and H₂O (10 ml) was stirred at 80° C. overnight under N₂. The resulting mixture was filtered, extracted with EA (100 ml) three times, and the organic phase was concentrated, purified on silica gel column flash chromatography (DCM/MeOH=10/1) to afford 3a (1 g, 100% yield) as yellow solid. LC/MS: [M+H]⁺:381.

Step 2: A mixture of 3a (200 mg, 0.526 mmol), 1-methyl-1H-pyrazol-3-amine (102 mg, 1.05 mmol), Brettphos Pd G₃ (48 mg, 0.053 mmol) and K₃PO₄ (167 mg, 0.789 mmol) in dioxane (20 mL) was stirred at 100° C. overnight under N₂. The resulting mixture was filtered, and the organic phase was concentrated, purified by Prep-HPLC to afford 14 (53.06 mg, 21% yield) as a yellow solid. LC/MS: [M+H]⁺: 442.1. ¹H NMR (400 MHz, DMSO) δ 10.41 (s, 1H), 8.73 (s, 1H), 8.33 (d, J=22.8 Hz, 2H), 7.66 (d, J=2.0 Hz, 1H), 6.45 (s, 1H), 4.55 (d, J=9.2 Hz, 2H), 4.26 (d, J=9.2 Hz, 3H), 3.68 (s, 4H), 3.21-3.26 (m, 3H), 2.36 (s, 3H), 1.24 (t, J=7.2 Hz, 3H).

### Example 15: Preparation of (R)-3-cyclopentyl-3-(4-(5-methyl-2-((1-(2-morpholino-2-oxoethyl)-1H-pyrazol-4-yl)amino)pyrimidin-4-yl)-1H-pyrazol-1-yl)propanenitrile (15)

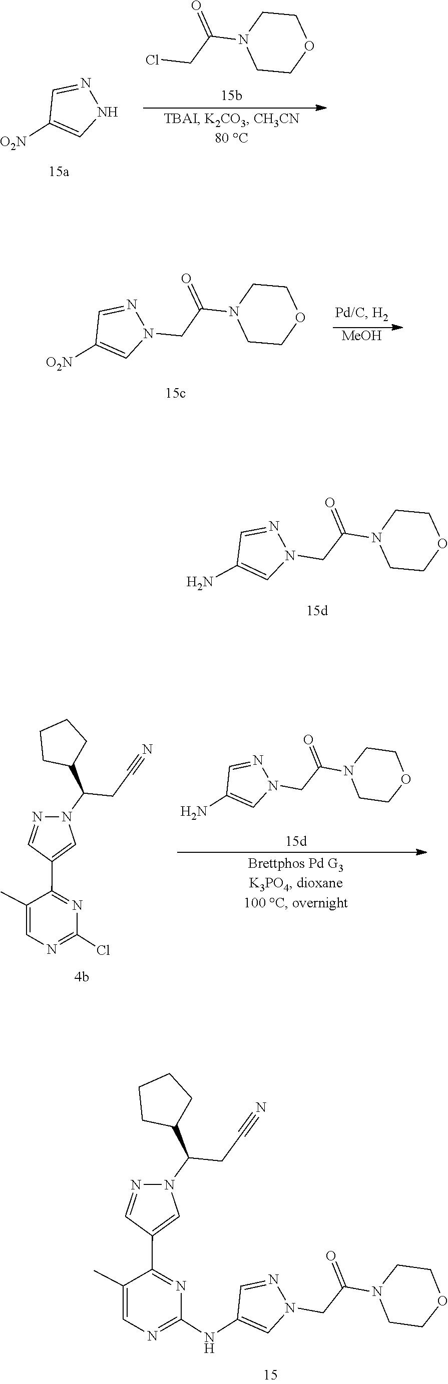

Step 1: A mixture of 15a (280 mg, 2.48 mmol), 15b (808 mg, 4.94 mmol), K₂CO₃ (1.02 g, 7.38 mmol) and TBAI (183 mg, 0.49 mmol) in MeCN (10 mL) was stirred at 80° C. for 2 h. The mixture was concentrated, purified on silica gel column flash chromatography (PE/EA=3/1) to afford 15c (500 mg, 83.9%) as brown solid. LC/MS: [M+H]⁺:241.

Step 2: A mixture of 15c (500 mg, 2.08 mmol), Pd/C (50 mg) in MeOH (10 mL) was stirred at 50° C. for 2 h under H₂. The resulting mixture was filtered. The filtrate was concentrated and purified on silica gel column flash chromatography (PE/EA=1/1) to afford 15d (400 mg, 91.5% yield) as brown solid. LC/MS: [M+H]⁺:211.

Step 3: A mixture of 4b (170 mg, 0.54 mmol), 15d (136 mg, 0.65 mmol), K₃PO₄ (172 mg, 0.81 mmol) and Brettphos Pd G₃ (49 mg, 0.054) in dioxane (10 mL) was stirred at 100° C. overnight under N₂. The mixture was concentrated, purified by Prep-HPLC to afford 15 (16 mg, 6.1% yield) as a yellow solid. LC/MS: [M+H]⁺:490.2. ¹H NMR (400 MHz, DMSO) δ 9.21 (s, 1H), 8.48 (s, 1H), 8.24 (s, 1H), 8.17 (s, 1H), 7.93 (s, 1H), 7.50 (s, 1H), 5.11 (s, 2H), 4.55 (td, J=9.2, 4.8 Hz, 1H), 3.59 (dd, J=12.4, 4.8 Hz, 4H), 3.53-3.41 (m, 4H), 3.25-3.13 (m, 2H), 2.30 (s, 3H), 1.80 (dd, J=12.0, 7.2 Hz, 1H), 1.68-1.42 (m, 4H), 1.39-1.11 (m, 4H).

### Example 16: Preparation of (R)-3-cyclopentyl-3-(4-(2-((1-isopropyl-1H-pyrazol-4-yl)amino)-5-methylpyrimidin-4-yl)-1H-pyrazol-1-yl)propanenitrile (16)

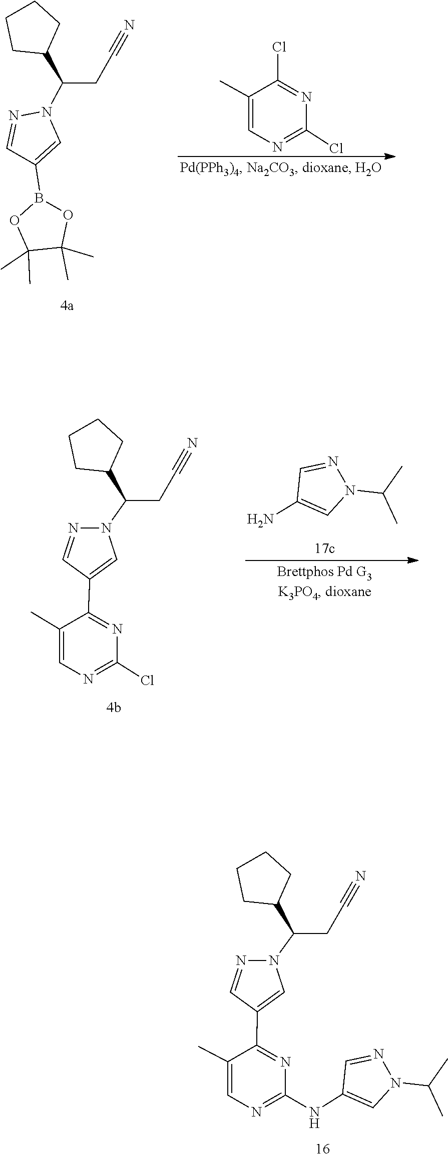

Step 1: To a solution of 4a (400 mg, 1.27 mmol), 2, 4-dichloro-5-methylpyrimidine (617 mg, 3.79 mmol), Na₂CO₃ (269 mg, 2.54 mmol) in dioxane/H₂O (30 ml/3 ml), Pd(PPh₃)₄ (150 mg, 0.127 mmol) was added, the mixture was stirred at 80° C. for 16 h under the N₂. Diluted it with EA, extracted with EA, concentrated and purified by SGC (PE:EA=2:1) afford 4b (530 mg, 100% yield) as yellow oil. LC/MS: [M+H]⁺:316.

Step 2: To a solution of 4b (170 mg, 0.54 mmol), 17c (81 mg, 0.648 mmol), K₃PO₄ (172 mg, 0.81 mmol) in dioxane (10 ml), Brettphos Pd G₃ (49 mg, 0.054 mmol) was added, the mixture was stirred at 100° C. under the N₂ for 16 h. Diluted and filtered, concentrated purified by SGC (DCM:MeOH=10:1) to afford 16 (20.02 mg, 9.18% yield) as a yellow solid. LC/MS: [M+H]⁺:405.3. 1H NMR (400 MHz, DMSO) δ 9.15 (s, 1H), 8.48 (s, 1H), 8.24 (s, 1H), 8.14 (s, 1H), 7.94 (s, 11H), 7.51 (s, 1H), 4.56 (td, J=9.6, 4.8 Hz, 11H), 4.46 (dd, J=13.2, 6.8 Hz, 1H), 2.46-2.36 (m, 1H), 2.29 (s, 3H), 1.82 (dd, J=11.6, 4.8 Hz, 1H), 1.64-1.50 (m, 3H), 1.39-1.41 (m, 6H), 1.38-1.12 (m, 4H).

### Example 17: Preparation of 2-(1-(ethylsulfonyl)-3-(4-(2-((1-isopropyl-1H-pyrazol-4-yl)amino)-5-methylpyrimidin-4-yl)-1H-pyrazol-1-yl)azetidin-3-yl)acetonitrile (17)

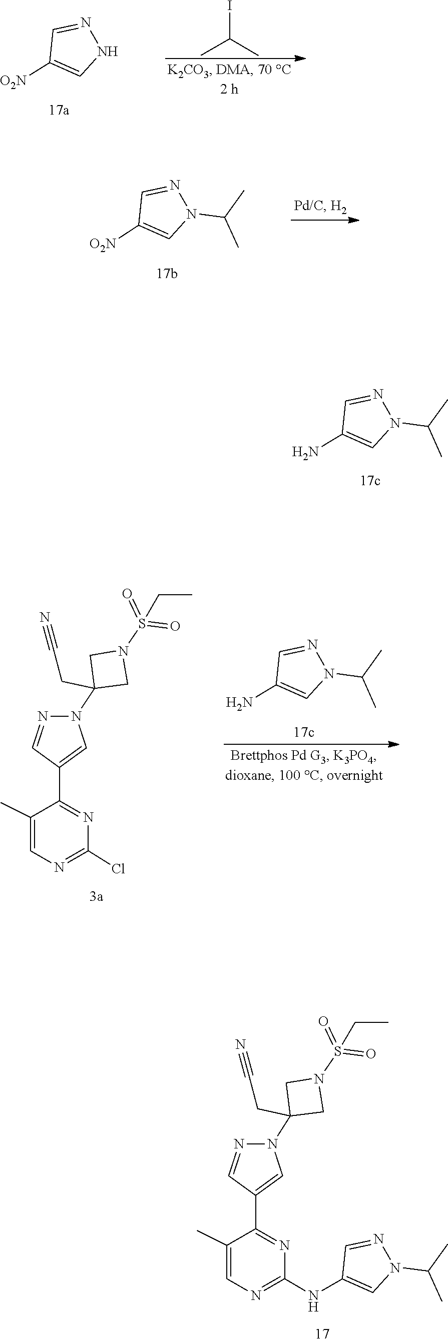

Step 1: To a solution of 17a (3 g, 26.5 mmol), 2-iodopropane (9 g, 53 mmol), K₂CO₃ (7.3 g, 53 mmol) in DMA (30 ml), the mixture was stirred at 70° C. for 2 h. Added EA (100 ml), washed by H₂O, Sat brine (100 ml), the organic phase was concentrated to afford 17b (4 g, 97.6% yield). LC/MS: [M+H]⁺:156.

Step 2: To a solution of 17b (2 g, 12.9 mmol) in MeOH (20 ml), Pd/C (400 mg) was added, the mixture was stirred under the H₂ at rt for 3 h. Diluted and filtered it, concentrated to afford 17c (1.8 g, 100% yield). LC/MS: [M+H]⁺:126.

Step 3: To a solution of 3a (150 mg, 0.395 mmol), 17c (59 mg 0.474 mmol), K₃PO₄ (126 mg, 0.592 mmol) in dioxane (10 ml), Brettphos Pd G₃ (36 mg, 0.04 mmol) was added, the mixture was stirred at 100° C. under the N₂ for 16 h. Diluted and filtered it, concentrated purified by SGC (DCM:MeOH=13:1) to afford 17 (27.9 mg, 15.1% yield) as a yellow solid. LC/MS: [M+H]⁺:470.3. ¹H NMR (400 MHz, DMSO) δ 9.20 (s, 1H), 8.61 (s, 1H), 8.26 (d, J=11.2 Hz, 2H), 7.95 (s, 1H), 7.50 (s, 1H), 4.56 (d, J=9.2 Hz, 2H), 4.48 (dd, J=13.2, 6.4 Hz, 1H), 4.24 (d, J=9.2 Hz, 2H), 3.67 (s, 2H), 3.22 (t, J=7.2 Hz, 2H), 2.32 (s, 3H), 1.41 (d, J=6.8 Hz, 6H), 1.24 (t, J=7.2 Hz, 3H).

### Example 18: Preparation of 2-(1-(ethylsulfonyl)-3-(4-(5-methyl-2-((1-methyl-1H-pyrazol-4-yl)amino)pyrimidin-4-yl)-1H-pyrazol-1-yl)azetidin-3-yl)acetonitrile (18)

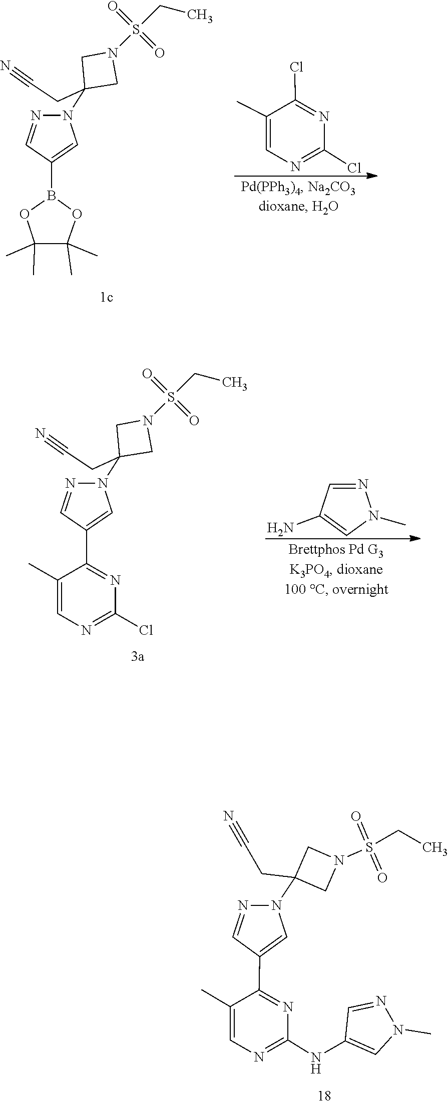

Step 1: A mixture of 1c (20.0 g, 52.63 mmol), 2, 4-dichloro-5-methylpyrimidine (25.6 g, 157.89 mmol), Pd(PPh₃)₄ (6.0 g) and Na₂CO₃ (22.3 g, 210.52 mmol) in dioxane (400 mL) and H₂O (50 ml) was stirred at 80° C. overnight under N₂. The resulting mixture was triturated with ethyl acetate, filtered. The filtered cake was triturated with water to afford 3a (16.1 g, 80.5% yield) as a light-yellow solid. LC/MS: [M+H]⁺:380.9.

Step 2: A mixture of 3a (16.1 g, 42.37 mmol), 1-methyl-1H-pyrazol-4-amine (12.3 g, 127.11 mmol), Brettphos Pd G₃ (3.8 g, 4.24 mmol) and K₃PO₄ (35.9 g, 169.47 mmol) in dioxane (300 mL) was stirred at 100° C. for 24 h under N₂. The reaction mixture was concentrated and triturated with DCM. After filtration, it was triturated with water to afford 18 (8.31 g, 44.4% yield) as a yellow solid. LCMS: Rt: 1.361 min; MS m/z (ESI): [M+H]⁺: 442.1. ¹H NMR (400 MHz, DMSO-d₆): δ 9.21 (s, 1H), 8.61 (s, 1H), 8.27 (s, 2H), 7.93 (s, 1H), 7.46 (s, 1H), 4.56 (d, J=9.2 Hz, 1H), 4.24 (d, J=9.2 Hz, 2H), 3.82 (s, 3H), 3.68 (s, 2H), 3.21-3.26 (m, 2H), 2.32 (s, 3H), 1.25 (t, J=7.2 Hz, 3H).

### Example 19: Preparation of 2-(1-(ethylsulfonyl)-3-(4-(5-methyl-2-((1-methyl-1H-1,2,3-triazol-4-yl)amino)pyrimidin-4-yl)-1H-pyrazol-1-yl)azetidin-3-yl)acetonitrile (19)

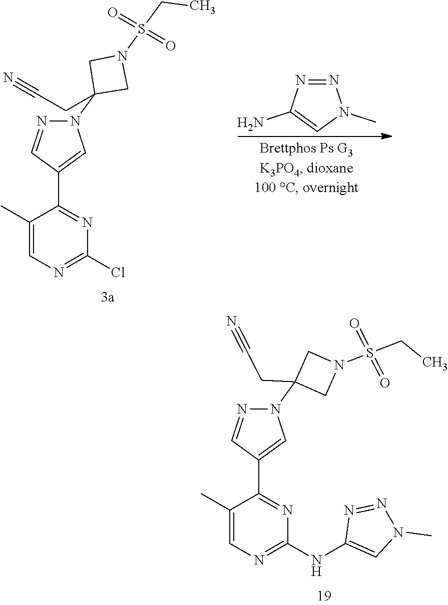

Step 1: A mixture of 3a (120 mg, 0.32 mmol), 1-methyl-1H-1,2,3-triazol-4-yl)amine (62 mg, 0.63 mmol), Brettphos Pd G₃ (10 mg) and K₃PO₄ (167 mg, 0.79 mmol) in dioxane (10 mL) was stirred at 110° C. overnight. The resulting mixture was filtered, and the residue was concentrated, purified by Prep-HPLC to afford TLL-117 (20.1 mg, 14%) as a light-yellow solid. LC/MS: [M+H]⁺:443.4. ¹H NMR (400 MHz, DMSO) δ 9.94 (s, 1H), 8.65 (s, 1H), 8.33 (s, 2H), 8.16 (s, 1H), 4.57 (d, J=9.1 Hz, 2H), 4.24 (d, J=9.1 Hz, 2H), 4.05 (s, 3H), 3.68 (s, 2H), 3.23 (dd, J=14.6, 7.3 Hz, 2H), 2.35 (s, 3H), 1.25 (t, J=7.3 Hz, 3H).

### Example 20: Preparation of N-((1s,3s)-3-(methyl(5-methyl-2-((1-methyl-1H-pyrazol-4-yl)amino)pyrimidin-4-yl)amino)cyclobutyl)propane-1-sulfonamide (20)

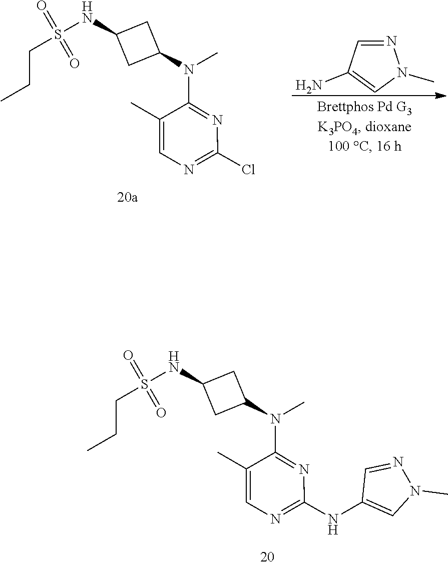

Step 1: A mixture of 20a (300 mg, 0.90 mmol), 1-methyl-1H-pyrazol-4-amine (175 mg, 1.81 mmol), Brettphos Pd G₃ (25 mg) and K₃PO₄ (478 mg, 2.26 mmol) in dioxane (20 mL) was stirred at 100° C. overnight. The resulting mixture was filtered, and the residue was concentrated, purified by Prep-HPLC to afford 20 (94.3 mg, 27%) as a light-yellow solid. LC/MS: [M+H]⁺:394.3. ¹H NMR (400 MHz, DMSO) δ 8.80 (s, 1H), 7.79 (s, 1H), 7.77 (s, 1H), 7.46 (s, 1H), 7.43 (s, 1H), 4.03-4.02 (m, 1H), 3.78 (s, 3H), 3.52-3.46 (m, 1H), 2.93-2.89 (m, 5H), 2.57-2.55 (m, 2H), 2.12-2.08 (m, 5H), 1.69-1.62 (m, 2H), 0.97 (t, J=7.6 Hz, 3H).

### Example 21: Preparation of N-methyl-1-((1r,4r)-4-(methyl(5-methyl-2-((1-methyl-1H-pyrazol-4-yl)amino)pyrimidin-4-yl)amino)cyclohexyl)methanesulfonamide (21)

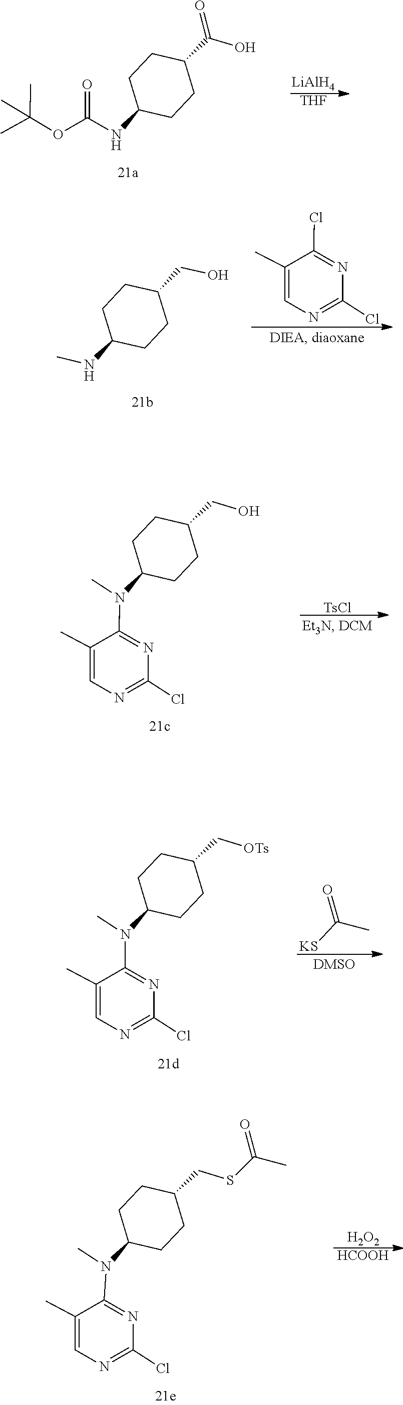

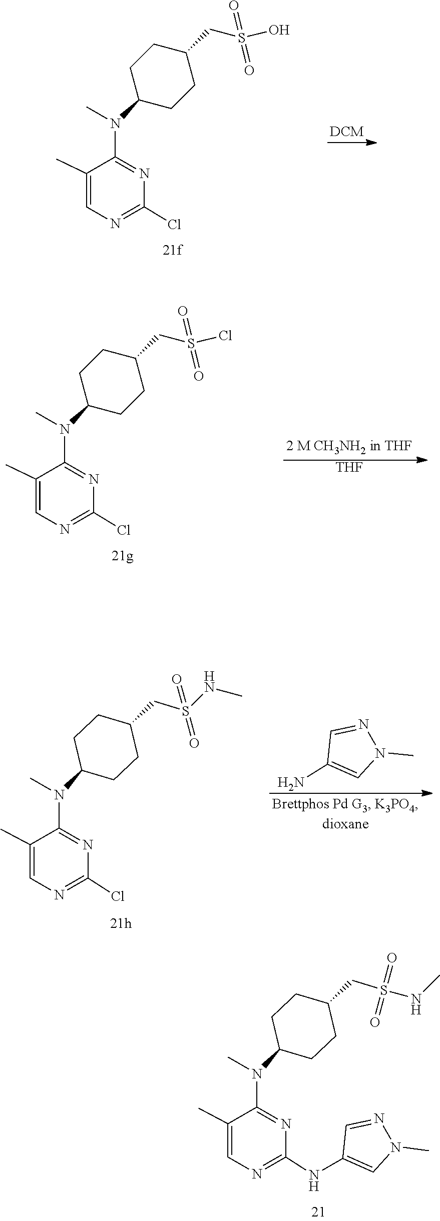

Step 1: To a mixture of 21a (5.0 g, 20.6 mmol) in THF (100 mL) was added LiAlH₄ (3.9 g, 103 mmol) at 0° C., the reaction mixture was stirred at 75° C. for 16 h. The reaction was quenched by H₂O (3.9 mL), 15% of NaOH (3.9*3 mL) and H₂O (3.9 mL). Subsequently, the resulting mixture was filtered and the filtrate was concentrated to afford 21b (3.1 g, >99%) as a yellow solid. LC/MS: [M+H]⁺:144.6.

Step 2: A mixture of 21b (2.6 g, 18.2 mmol), 2,4-dichloro-5-methylpyrimidine (8.9 g, 54.6 mmol) and DIEA (9.4 g, 72.8 mmol) in dioxane (100 mL) was stirred at 105° C. overnight, then cooled to room temperature. The resulting mixture was concentrated and the residue was purified on silica gel column flash chromatography (PE/EA=1/1) to afford 21c (1.9 g, 38%) as a yellow solid. LC/MS: [M+H]⁺:270.4.

Step 3: To a mixture of 21c (1.9 g, 7.1 mmol) and Et₃N (2.9 g, 28.2 mmol) in DCM (50 mL) was added TsCl (4.0 g, 21.3 mmol) at 0° C., the reaction mixture was stirred at rt for 16 h. The reaction was concentrated and the residue purified on silica gel column flash chromatography (PE/EA=1/1) to afford 21d (2.8 g, 93%) as a yellow solid. LC/MS: [M+H]⁺:424.1.

Step 4: A mixture of 21d (2.8 g, 6.6 mmol) and potassium ethanethioate (1.5 g, 13.2 mmol) in DMSO (50 mL) was stirred at 55° C. overnight, then cooled to room temperature. The resulting mixture was diluted with EA (50 mL) and washed with H₂O (20 mL*3), extracted with EA. The organic phase was washed with brine, dried over Na₂SO₄, and filtered. The filtrate was concentrated and the residue was purified on silica gel column flash chromatography (PE/EA=1/1) to afford 21e (1.0 g, 46%) as yellow solid. LC/MS: [M+H]⁺: 328.4.

Step 5: To a mixture of 21e (800 mg, 2.4 mmol) in HCOOH (2 mL) was added H₂O₂ (408 mg, 12.0 mmol). The reaction mixture was stirred at rt for 16 h. The reaction was quenched by Na₂S₂O₃, and the residue was purified by flash chromatography (0.1% TFA) to afford 21f (350 mg, 42%) as a white solid. LC/MS: [M+H]⁺: 334.4.

Step 6: To a mixture of 21f (350 mg, 1.1 mmol) in DCM (10 mL) was added DMF (two drops) and SOCl₂ (625 mg, 5.5 mmol). The reaction mixture was stirred at 45° C. for 2 h. The reaction was concentrated to afford 21g (385 mg, >99%) as a yellow oil. LC/MS: [M+H]⁺:352.3.

Step 7: A mixture of 21 g (385 mg, 1.1 mmol) and 2 M CH₃NH₂ in THF (2.75 mL, 5.5 mmol) in THF (5 mL) was stirred at rt overnight. The resulting mixture was concentrated and the residue was purified on silica gel column flash chromatography (DCM/MeOH=20/1) to afford 21h (300 mg, 79%) as a yellow solid. LC/MS: [M+H]⁺:347.1.

Step 8: A mixture of 21h (150 mg, 0.43 mmol), 1-methyl-1H-pyrazol-4-amine (84 mg, 0.86 mmol), Brettphos Pd G₃ (10 mg) and K₃PO₄ (228 mg, 1.1 mmol) in dioxane (10 mL) was stirred at 100° C. overnight. The resulting mixture was filtered, and the residue was concentrated, purified by Prep-HPLC to afford 21 (55 mg, 31%) as a white solid. LC/MS: [M+H]⁺:408.1. ¹H NMR (400 MHz, DMSO-d₆): δ 10.21 (s, 1H), 7.81 (s, 1H), 7.70 (s, 1H), 7.52 (s, 1H), 6.90 (d, J=4.8 Hz, 1H), 4.33-4.32 (m, 1H), 3.84 (s, 3H), 3.12 (s, 3H), 2.94 (d, J=6.4 Hz, 2H), 2.58 (d, J=4.4 Hz, 3H), 2.25 (s, 3H), 2.07-2.04 (m, 2H), 1.86-1.84 (m, 1H), 1.72-1.69 (m, 4H), 1.26-1.16 (m, 2H).

### Example 22: Preparation of 3-((3R,4R)-4-methyl-3-(methyl(5-methyl-2-((1-methyl-1H-pyrazol-4-yl)amino)pyrimidin-4-yl)amino)piperidin-1-yl)-3-oxopropanenitrile (22)

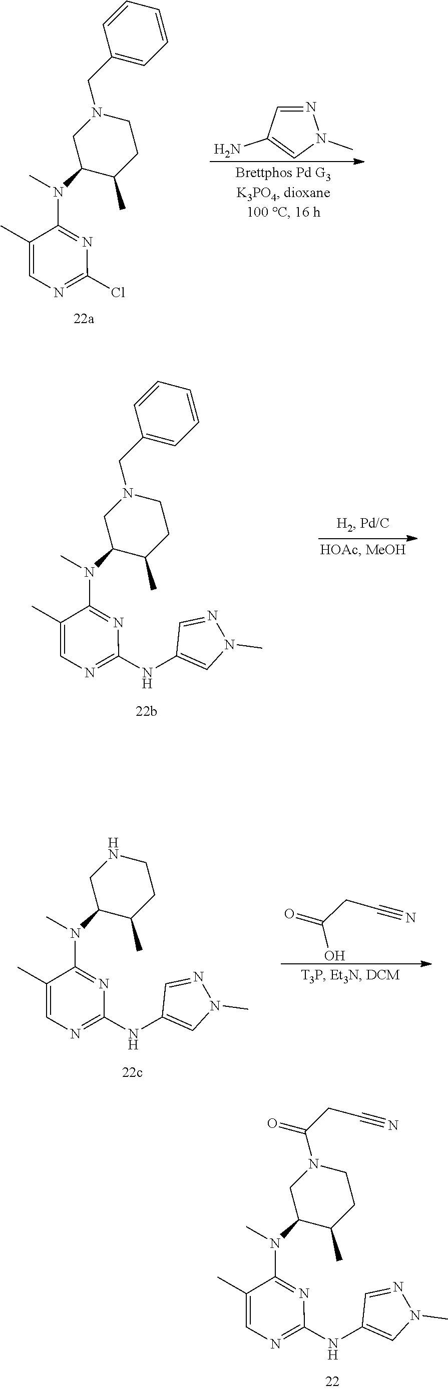

Step 1: A mixture of 22a (600 mg, 1.74 mmol), 1-methyl-1H-pyrazol-4-amine (338 mg, 3.48 mmol), Brettphos Pd G₃ (10 mg) and K₃PO₄ (922 mg, 4.35 mmol) in dioxane (10 mL) was stirred at 100° C. overnight. The resulting mixture was filtered, and the residue was concentrated, purified on silica gel column flash chromatography (DCM/MeOH=10/1) to afford 22b (800 mg, >99%) as a brown solid. LC/MS: [M+H]⁺:406.2.

Step 2: To a mixture of 22b (405 mg, 1.0 mmol) in MeOH (10 mL) was added Pd/C (200 mg) and HOAc (10 drops), the reaction mixture was stirred at 50° C. for 16 h. The resulting mixture was filtered, and the residue was concentrated, purified on silica gel column flash chromatography (DCM/MeOH=10/1) to afford 22c (140 mg, 44%) as a brown solid. LC/MS: [M+H]⁺:316.2.

Step 3: To a mixture of 22c (100 mg, 0.32 mmol) and 2-cyanoacetic acid (54 mg, 0.64 mmol) in DCM (10 mL) was added Et₃N (97 mg, 0.96 mmol) and T₃P (204 mg, 0.64 mmol). The reaction mixture was stirred at rt for 16 h. The resulting mixture was filtered, and the residue was concentrated, purified by Prep-HPLC to afford 22 (19.4 mg, 16%) as white solid. LC/MS: [M+H]⁺: 383.3. ¹H NMR (400 MHz, DMSO-d₆): δ 8.75-8.74 (m, 1H), 7.80 (d, J=7.2 Hz, 1H), 7.72 (s, 1H), 7.42-7.38 (m, 1H), 4.23-4.02 (m, 1H), 3.86 (d, J=4.0 Hz, 2H), 3.83-3.82 (m, 1H), 3.77 (d, J=3.6 Hz, 3H), 3.74-3.70 (m, 1H), 3.59-3.14 (m, 2H), 2.96 (s, 3H), 2.51-2.50 (m, 1H), 2.12 (d, J=6.0 Hz, 3H), 1.76-1.51 (m, 2H), 1.00 (t, J=6.8 Hz, 3H).

### Example 23: Preparation of (R)-3-cyclopentyl-3-(4-(5-methyl-2-((1-methyl-1H-benzo[d]imidazol-6-yl)amino)pyrimidin-4-yl)-1H-pyrazol-1-yl)propanenitrile (23)

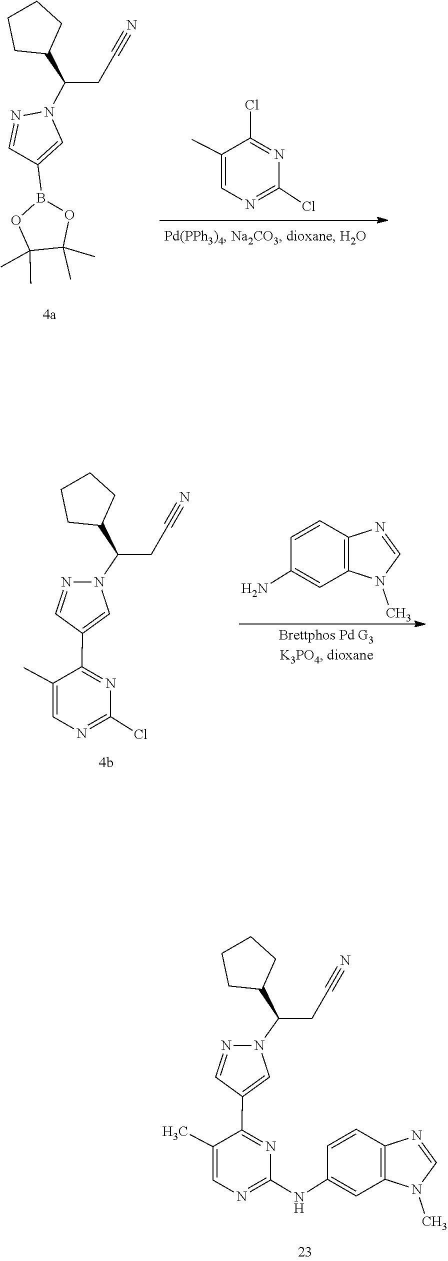

Step 1: To a flask containing dioxane (600 mL) and water (60 mL) was added 4a (25 g, 79.3 mmol), 2, 4-dichloro-5-methylpyrimidine (38.8 g, 238 mmol), and Na₂CO₃ (25.2 g, 238 mmol) at room temperature. Pd(PPh₃)₄ (9 g, 23.8 mmol) was added quickly. The resulting mixture was purged with nitrogen 3 times and the mixture was stirred at 80° C. for 18 h under N₂. The reaction mixture was filtered and the filtrate was concentrated. The crude residue was purified by column chromatography to obtain 4b (20 g, 80% yield) as a yellow oil. LC/MS: [M+H]⁺:316.

Step 2: To a flask containing dioxane (500 ml) was added 4b (10 g, 31.7 mmol), 1-methyl-1H-benzo[d]imidazol-6-amine (14 g, 95.1 mmol), and K₃PO₄ (33.6 g, 158.5 mmol) at room temperature. Brettphos Pd G₃ (2.88 g, 3.17 mmol) was added quickly. The resulting mixture was purged with nitrogen 3 times and the mixture was stirred at 110° C. for 48 h under nitrogen atmosphere. The reaction mixture was filtered and the filtrate was concentrated. The crude residue was purified by column chromatography (DCM:MeOH=30:1) to obtain an impure product. The residue was purified again by Prep-HPLC to obtain (23) (5 g, Yield=37%) as a red solid. ¹H NMR (400 MHz, DMSO-d₆): δ 9.47 (s, 1H), 8.55 (s, 1H), 8.36 (d, J=1.6 Hz, 1H), 8.33 (s, 1H), 8.22 (s, 1H), 8.04 (s, 1H), 7.54-7.51 (m, 1H), 7.38-7.35 (m, 1H), 4.59-4.55 (m, 1H), 3.81 (s, 3H), 3.29-3.16 (m, 2H), 2.43-2.40 (m, 1H), 2.34 (s, 3H), 1.83-1.80 (m, 1H), 1.63-1.44 (m, 4H), 1.35-1.18 (m, 3H). LCMS: Rt: 1.143 min; MS m/z (ESI): 427.3[M+H]⁺.

### Example 24: Preparation of (R)-3-cyclopentyl-3-(4-(2-((1-(2-hydroxyethyl)-1H-pyrazol-4-yl)amino)-5-methylpyrimidin-4-yl)-1H-pyrazol-1-yl)propanenitrile (24)

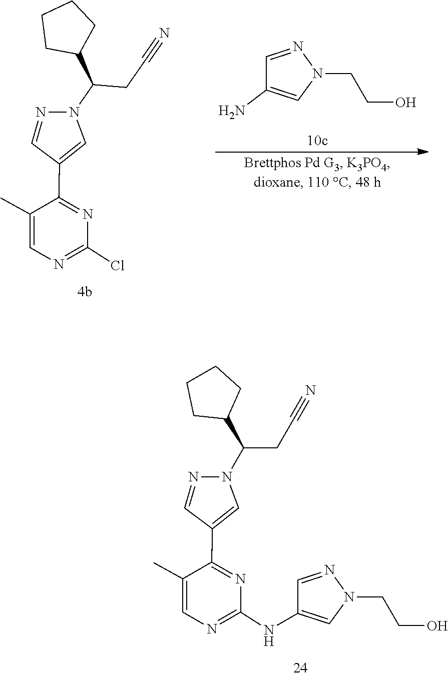 [SMILES: Cc1cnc(Cl)nc1-c1cnn([C@H](CC#N)C2CCCC2)c1 ; Nc1cnn(CCO)c1 ; Cc1cnc(Nc2cnn(CCO)c2)nc1-c1cnn([C@H](CC#N)C2CCCC2)c1]

Step 1: To a flask containing dioxane (500 ml) was added 4b (10 g, 31.7 mmol), 10c (12 g, 95.1 mmol), and K₃PO₄ (33.6 g, 158.5 mmol) at room temperature. Brettphos Pd G₃ (2.88 g, 3.17 mmol) was added quickly. The resulting mixture was purged with nitrogen 3 times and the mixture was stirred at 110° C. for 48 h under nitrogen atmosphere. The reaction mixture was filtered and the filtrate was concentrated. The crude residue was purified by column chromatography (DCM:MeOH=50:1) to obtain impure product. The impure product was triturated with methanol to obtain (24) (4.52 g, Yield=35%) as a light-brown solid. ¹H NMR (400 MHz, DMSO-d₆): δ 9.15 (s, 1H), 8.47 (s, 1H), 8.24 (s, 1H), 8.17 (s, 1H), 7.98 (s, 1H), 7.50 (s, 1H), 4.91 (t, J=4.8 Hz, 1 H), 4.57-4.53 (m, 1H), 4.12 (t, J=5.2 Hz, 2H), 3.75-3.71 (m, 2H), 3.32-3.18 (m, 2H), 2.42-2.40 (m, 1H), 2.29 (s, 3H), 1.83-1.80 (m, 1H), 1.62-1.32 (m, 4H), 1.29-1.18 (m, 3H). LCMS: Rt: 1.407 min; MS m/z (ESI): [M+H]⁺:407.2.

Compound 25, 26, and 27 are prepared by procedures previously described, as in the preparation of compound 13, 18 and 23.

### Example 1b. TYK2, JAK1, JAK2, JAK3 Biochemical Tests

The tests were conducted by Reaction Biology Corp, Malvern, PA (Anastassiadis et al. Nat Biotechnol. 2011; 29(11):1039-45). The step is briefly described as follows.

### Reagents:

Basic reaction buffer: 20 mM Hepes (pH 7.5), 10 mM MgCl₂, 1 mM EGTA, 0.02% Brij35, 0.02 mg/ml BSA, 0.1 mM Na₃VO₄, 2 mM DTT and 1% DMSO. The required cofactor was added to each kinase reaction respectively.

### Reaction Steps:

1. Preparing the designated substrate in the newly prepared basic reaction buffer;
2. Transferring the required cofactor to the above matrix solution;
3. Transferring the designated kinase to the substrate solution and mixing well slightly;
4. Transferring a compound of formulae I-III in DMSO to the kinase reaction mixture with Acoustic technique (Echo550; nanoliter range), culturing for 20 minutes at room temperature;
5. Introducing ³³P-ATP (specific activity: 10 μCi/μl) to the reaction mixture to trigger a reaction;
6. Culturing at room temperature and conducting a kinase reaction for 2 hours;
7. Plotting the reaction on P81 ion exchange paper;
8. Testing the kinase activity with a filter binding assay.

The test results as shown below (IC₅₀ in nM):

| compound | TYK2 | JAK1 | JAK2 | JAK3 |
|---|---|---|---|---|
| 2 | <1 | <10 | <1 | |
| 4 | <1 | <1 | <1 | |
| 5 | <1 | <1 | <1 | |
| 6 | <1 | <1 | <1 | |
| 7 | <1 | <1 | <1 | |
| 8 | <1 | <1 | <1 | |
| 9 | <1 | <1 | <1 | |
| 11 | <1 | | | |
| 14 | >10 | >10 | | |
| 18 | <1 | <1 | <1 | >10 |
| 19 | >10 | >10 | >10 | |
| 20 | >10 | >10 | >10 | |
| 21 | >100 | >100 | >100 | |
| 22 | >100 | >100 | | |
| 23 | <1 | <1 | <1 | |
| 24 | <1 | <1 | <1 | |
| 26 | | <1 | | |

The above data shows that compounds of Formula II and III (examples 2, 4, 5, 6, 7, 8, 9, 11, 18, 23, 24, 26) are potent TYK2/JAK1/JAK2 inhibitors. In comparison, the 2-((pyrazol-3-yl)amino) compound (example 14, D2) and 2-((triazol-4-yl)amino) compound (example 19) are >10× less potent than its 2-((pyrazol-4-yl)amino) analog (example 18). Compounds using the 4-pyrimidine substituents based on abrocitinib (example 20), tofacitinib (example 22), and oclacitinib (example 21) are >10×, >100×, >100×, respectively, less potent TYK2/JAK1/JAK2 inhibitors compared to their analogs of Formula II (example 4) and Formular III (example 18).

### Example 2b. TYK2 Cellular ELISA Assay Using NK92 Cells

Purpose: test cellular TYK2 activity using IL-12 and IL-18 stimulated IFNγ excretion assay in NK92 cells.

### Procedures:

1. Replace NK92 cell media with IL-2 by media without IL-2, maintain overnight;
2. Plate NK92 cells to 96-well plate at density of ~150,000 cell/well;
3. Add different concentrations of test article to wells, maintain in incubator at 37° C. and 5% CO2 for 1 hour;
4. Add IL-12 (final concentration: 2 ng/ml) and IL-18 (final concentration: 5 ng/ml) to stimulate for 24 hours in the above incubator;
5. After 24 hours, centrifuge at 2000 rpm for 5 min, take the supernatant and analyze according to instructions of the ELISA kit.

In this assay, compound 4 had IC₅₀<10 nM.

### Example 3b. JAK1 Cellular ELISA Assay Using PBMC Cells

Purpose: test cellular JAK1 activity using the IL-6 stimulated STAT3 phosphorylation assay in PBMC cells.

### Procedures:

1. Plate PBMC cells to 96-well plate at density of ~300,000 cell/well;
2. Add different concentrations of test article to wells, maintain in incubator at 37° C. and 5% CO2 for 1 hour;
3. Add IL-6 (final concentration: 100 ng/ml) to stimulate for 25 min in the above incubator;
4. After stimulation, transfer the suspension to 1.5 mL tube, centrifuge at 200 g for 5 min, collect cells, add 100 μL 1× lysate, lyse on ice for 1 hour;
5. Centrifuge at 4° C. and 12000 rpm for 10 min, take the supernatant and analyze according to instructions of the ELISA kit.

In this assay, compounds 4, 18, 23, and 24 had IC₅₀<500 nM.

### Example 4b. Human Liver Microsome Stability Assay

The assay was conducted following procedures as below:
1. Prepared 8 sample plates of 96-well plates, named as T0, T3, T6, T9, T15, T30, NCF30, BLANK.
2. Dispensed 100 μl/well human microsomes to each plate.
3. Dispensed 2 μL compound or control working solution/well to each plate (T0, T3, T6, T9, T15, T30, NCF30) except matrix blank and incubation plates at 37° C. for about 10 min. The final concentration of test compound was 1 μM. For T0, added 600 (μL/well) of cold stop solution immediately.
4. Added 98 μL 100 mM potassium phosphate buffer/well to NCF30 plate, incubated at 37° C., start reaction.
5. After pre-warming, dispensed NADPH regenerating system to 96-well plate as reservoir according to the plate map. Then added 98 μL/well to every plate (Blank, T30, T15, T9, T6, T3, T0) to start reaction.
6. The reaction was stopped by the addition of 600 μl of cold stop solution at 0, 3, 6, 9, 15 and 30 minutes. Samples were shaked for 10 min and then centrifuged at 3220 g for 20 min. The supernatant was taken from each well and diluted with dilution solution before being analyzed by LC/MS/MS.
7. The analyte/internal standard peak area ratios were converted to percentage remaining (% Remaining) with the following equation:

$$Remainig \% = \frac{Peak area ratio of analyte to internal standard at each time point}{Peak area ratio of analyte to internal standard at t = 0} \times 100 \%$$

The slope value, k, was determined by linear regression of the natural logarithm of the remaining percentage of the parent drug vs. incubation time curve. The in vitro t_{1/2} was determined from the slope value:

In vitro t_{1/2}=−(0.693/k)

Conversion of the in vitro t_{1/2} (min) into the in vitro intrinsic clearance (in vitro CLᵢₙₜ, μl/min/mg protein) was done using the following equation:

CL_{int (mic)}=0.693/t_{1/2}/mg microsome protein/ml

In this assay, the in vitro CL_{int(mic)} of compound 4 in human liver microsome is 65.9 μl/min/mg protein, and t_{1/2} is 21 min.

### Example 5b. Mini-Pig Dermal Pharmacokinetic Study

The aim of this study was to obtain the distribution of test articles in mini-pig after topical administration. Test articles were formulated as ointment and then applied to 2 female Guangxi Bama mini-pigs (8-12 kg) (Wujiang Tian yu Biological Technology). Briefly, total area estimated to be approximately 10% of the total body surface area were clipped free of hair before treatment using electric clippers. The test article was dosed evenly to the skin with 15 mg/cm² at 0 h, 8 h and 24 h. Test articles were removed before next dosing. Blood and skin samples were collected at 24 h (pre-dosing), 25 h, 28 h, 32 h, 48 h and 96 h. The blood was taken via peripheral vein of each animal into pre-chilled tube containing potassium (K2) EDTA as anticoagulant. Exposed skin was washed using ethanol and then wiped with dry cotton swab before skin sample collection. Eight samples of dosing skin were drilled with a biopsy punch (0.7 cm diameter) for each time point. Epidermis and dermis were separated and the eight samples from the same time point were pooled together and homogenized. All the above samples and homogenate were quantified by LC-MS/MS analysis.

This study demonstrated minimal systemic exposure and enrichment in dermis and epidermis of compound 4 when topically administered.

Exemplary results as shown below:

| | | | | Dermis/ | Epidermis/ |
|---|---|---|---|---|---|
| | Plasma | Dermis | Epidermis | Plasma | Plasma |
| | AUC₀₋ₗₐₛₜ | AUC₀₋ₗₐₛₜ | AUC₀₋ₗₐₛₜ | ratio | ratio |
| Example | (h*ng/ml) | (h*ng/ml) | (h*ng/ml) | (AUC) | (AUC) |
| Compound 4 | 103 | 517348 | 3523449 | 5023× | 34208× |

### Example 6b. Efficacy Study in hIL-23 Induced Psoriasis Model

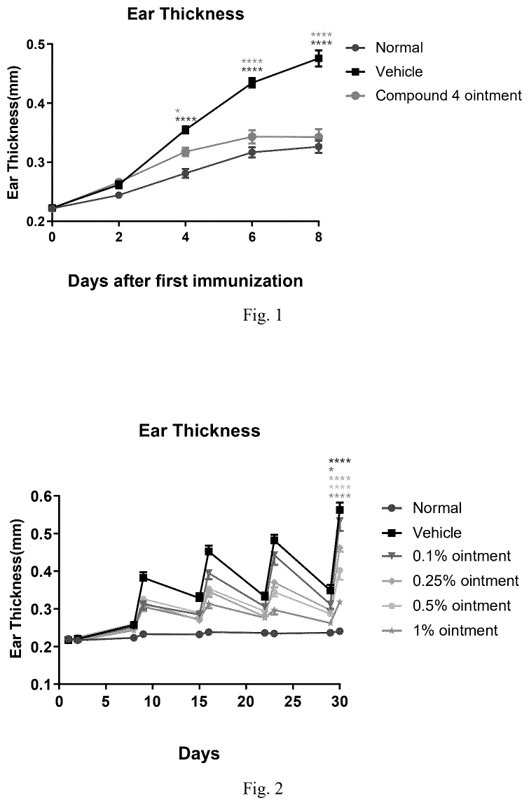

C57BL/6J mice (Beijing Vital River Laboratory) were randomized into normal, vehicle and treatment groups according to bodyweight and ear thickness on day 0. Immunization was conducted using hIL-23 (Sigma) in vehicle and treatment groups to induce a psoriasis-like inflammation, whereas PBS was used as a parallel in normal group. Human IL-23 immunized animals were topically administered vehicle and test articles, respectively, once-daily from day 1 to day 9. Ear thickness was assessed using a digital micrometer (QuantuMike, 293-185) before hIL-23 or PBS injection on day 0, 2, 4, 6, 8 and 10. Ear thickness was used to evaluate the efficacy of test article. Data are shown as Mean±SEM, using two-way ANOVA statistical analysis, *p<0.05, ****p<0.0001 compared with vehicle group, as shown in FIG. 1.

### Example 7b. Efficacy Study in 2, 4-Dinitro-Fluorobenzene (DNFB) Induced Atopic Dermatitis Model

BALB/c mice (Beijing Vital River Laboratory) of approximately 8 weeks old were randomized into normal, vehicle and treatment groups according to bodyweight and ear thickness at study initiation. Animals in vehicle and treatment groups received 0.15% DNFB stimulation on day 1, 8, 15, 22 and 29, and were once-daily administered with vehicle and test articles (0.1%, 0.25%, 0.5% and 1% ointment of compound 4), respectively. Ear thickness was evaluated using a digital micrometer (QuantuMike, 293-185) right before and 24 h after each DNFB stimulation.

Data were plotted as Mean±SEM, using two-way ANOVA statistical analysis, *p<0.05, ****p<0.0001 compared with vehicle group, as shown in FIG. 2.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 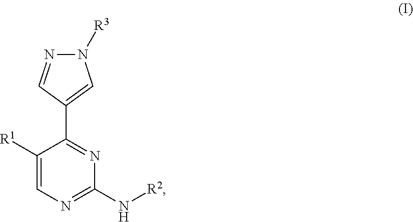
or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R³ is 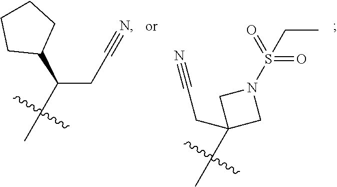 [SMILES: *[C@H](CC#N)C1CCCC1 ; *C1(CC#N)CN(S(=O)(=O)CC)C1]
R² is 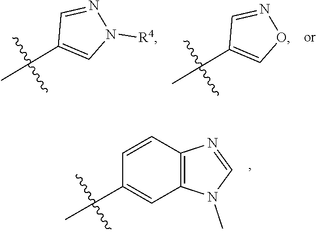
wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

2. A compound of Formula (II): 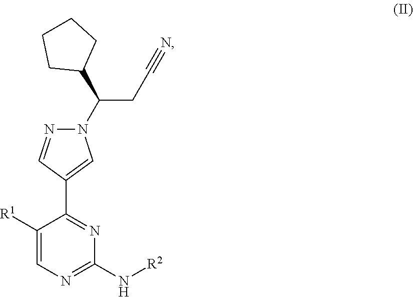
or a salt, solvate, hydrate, polymorph, co-crystal, tautomer, stereoisomer, isotopically labeled derivative, or prodrug thereof, wherein:
R¹ is C₁₋₃ alkyl, C₃₋₄ cycloalkyl, or halo;
R² is 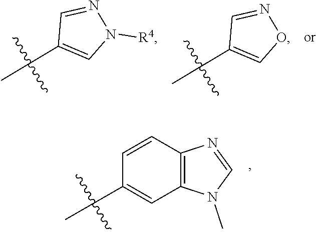
wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

3. A compound of Formula (III): 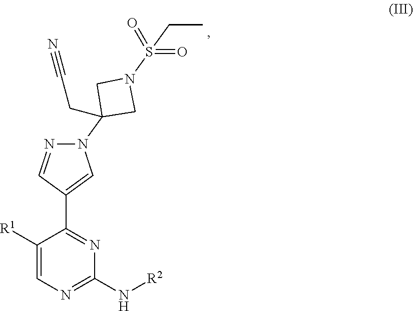
or a salt, solvate, hydrate, polymorph, co-crystal, tautomer, stereoisomer, isotopically labeled derivative, or prodrug thereof, wherein:
R¹ is C₁₋₃ alky, C₃₋₄ cycloalkyl, or halo;
R² is 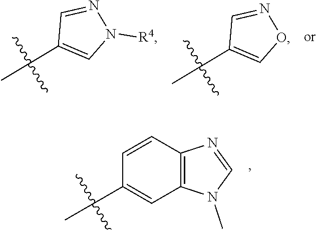
wherein: R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN.

4. The compound according to claim 1 or a pharmaceutically acceptable salt thereof, wherein: R² is 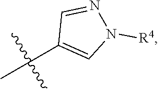
wherein R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN;
or, wherein: R² is 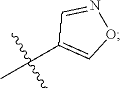 [SMILES: *c1cnoc1]
or, wherein R² is

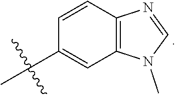 [SMILES: *c1ccc2ncn(C)c2c1]

5. (canceled)

6. (canceled)

7. The compound according to claim 1, or a pharmaceutically acceptable salt thereof, wherein: R¹ is C₁₋₃ alky, C₃₋₄ cycloalkyl, or halo.

8. (canceled)

9. The compound according to claim 1, or a pharmaceutically acceptable salt thereof, wherein: R¹ is CH₃ or Cl.

10. (canceled)

11. (canceled)

12. (canceled)

13. The compound according to claim 1, or a pharmaceutically acceptable salt thereof, wherein:
R⁴ is C₁₋₃ alkyl, optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN;
or, wherein: R⁴ is CH₂R⁵, R⁵ is 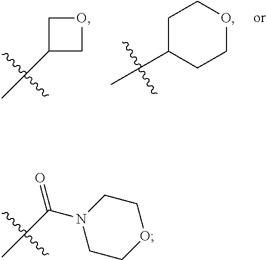 [SMILES: *C1COC1 ; *C1CCOCC1 ; "or" ; *C(=O)N1CCOCC1]
or, wherein: R⁴ is 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′,
R, R′ each is independently H, or C₁₋₃ alkyl optionally substituted with 1, 2, or 3 substituents independently selected from the group consisting of halo, OH, and CN;
or, wherein R⁴ is

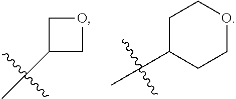 [SMILES: *C1COC1 ; *C1CCOCC1]

14. (canceled)

15. (canceled)

16. (canceled)

17. The compound according to claim 1, or a pharmaceutically acceptable salt thereof, wherein the compound is:
2-(3-(4-(5-cyclopropyl-2-(isoxazol-4-ylamino)pyrimidin-4-yl)-1H-pyrazol-1-yl)-1-(ethylsulfonyl) azetidin-3-yl) acetonitrile (1);
2-(3-(4-(5-cyclopropyl-2-((1-methyl-1H-pyrazol-4-yl) amino) pyrimidin-4-yl)-1H-pyrazol-1-yl)-1-(ethylsulfonyl) azetidin-3-yl) acetonitrile (2);
2-(1-(ethylsulfonyl)-3-(4-(2-(isoxazol-4-ylamino)-5-methylpyrimidin-4-yl)-1H-pyrazol-1-yl) azetidin-3-yl) acetonitrile (3);
2-(1-(ethylsulfonyl)-3-(4-(2-((1-(2-hydroxyethyl)-1H-pyrazol-4-yl)amino)-5-methylpyrimidin-4-yl)-1H-pyrazol-1-yl)azetidin-3-yl)acetonitrile (12);
2-(3-(4-(5-chloro-2-((1-(2-hydroxyethyl)-1H-pyrazol-4-yl)amino)pyrimidin-4-yl)-1H-pyrazol-1-yl)-1-(ethylsulfonyl)azetidin-3-yl)acetonitrile (13);
2-(1-(ethylsulfonyl)-3-(4-(2-((1-isopropyl-1H-pyrazol-4-yl)amino)-5-methylpyrimidin-4-yl)-1H-pyrazol-1-yl)azetidin-3-yl)acetonitrile (17);
2-(1-(ethylsulfonyl)-3-(4-(5-methyl-2-((1-methyl-1H-pyrazol-4-yl)amino)pyrimidin-4-yl)-1H-pyrazol-1-yl)azetidin-3-yl)acetonitrile (18);
2-(1-(ethylsulfonyl)-3-(4-(5-methyl-2-((1-methyl-1H-benzo[d]imidazol-6-yl)amino)pyrimidin-4-yl)-1H-pyrazol-1-yl)azetidin-3-yl)acetonitrile (26);
2-(3-(4-(5-chloro-2-((1-methyl-1H-benzol[d]imidazol-6-yl)amino)pyrimidin-4-yl)-1H-pyrazol-1-yl)-1-(ethylsulfonyl)azetidin-3-yl)acetonitrile (27).

18. The compound according to claim 1, f 2, or a pharmaceutically acceptable salt thereof, wherein the compound is:
(R)-3-cyclopentyl-3-(4-(5-methyl-2-((1-methyl-1H-pyrazol-4-yl) amino) pyrimidin-4-yl)-1H-pyrazol-1-yl) propanenitrile (4);
(R)-3-cyclopentyl-3-(4-(2-(isoxazol-4-ylamino)-5-methylpyrimidin-4-yl)-1H-pyrazol-1-yl) propanenitrile (5);
(R)-3-cyclopentyl-3-(4-(5-methyl-2-((1-((tetrahydro-2H-pyran-4-yl) methyl)-1H-pyrazol-4-yl) amino) pyrimidin-4-yl)-1H-pyrazol-1-yl)propanenitrile (6);
(R)-3-cyclopentyl-3-(4-(5-methyl-2-((1-(tetrahydro-2H-pyran-4-yl)-1H-pyrazol-4-yl) amino) pyrimidin-4-yl)-1H-pyrazol-1-yl)propanenitrile (7);
(R)-3-cyclopentyl-3-(4-(5-methyl-2-((1-(oxetan-3-yl)-1H-pyrazol-4-yl) amino) pyrimidin-4-yl)-1H-pyrazol-1-yl) propanenitrile (8);
(R)-3-cyclopentyl-3-(4-(5-methyl-2-((1-(oxetan-3-ylmethyl)-1H-pyrazol-4-yl) amino) pyrimidin-4-yl)-1H-pyrazol-1-yl) propanenitrile (9);
(R)-3-(4-(5-chloro-2-((1-(2-hydroxyethyl)-1H-pyrazol-4-yl)amino)pyrimidin-4-yl)-1H-pyrazol-1-yl)-3-cyclopentylpropanenitrile (10);
((R)-3-(4-(5-chloro-2-((1-(tetrahydro-2H-pyran-4-yl)-1H-pyrazol-4-yl)amino)pyrimidin-4-yl)-1H-pyrazol-1-yl)-3-cyclopentylpropanenitrile (11);
(R)-3-cyclopentyl-3-(4-(5-methyl-2-((1-(2-morpholino-2-oxoethyl)-1H-pyrazol-4-yl)amino)pyrimidin-4-yl)-1H-pyrazol-1-yl)propanenitrile (15);
(R)-3-cyclopentyl-3-(4-(2-((1-isopropyl-1H-pyrazol-4-yl)amino)-5-methylpyrimidin-4-yl)-1H-pyrazol-1-yl)propanenitrile (16);
(R)-3-cyclopentyl-3-(4-(5-methyl-2-((1-methyl-1H-benzo[d]imidazol-6-yl)amino)pyrimidin-4-yl)-1H-pyrazol-1-yl)propanenitrile (23);
(R)-3-cyclopentyl-3-(4-(2-((1-(2-hydroxyethyl)-1H-pyrazol-4-yl)amino)-5-methylpyrimidin-4-yl)-1H-pyrazol-1-yl)propanenitrile (24);
(R)-3-(4-(5-chloro-2-((1-methyl-1H-benzo[d]imidazol-6-yl)amino)pyrimidin-4-yl)-1H-pyrazol-1-yl)-3-cyclopentylpropanenitrile (25).

19. The compound according to claim 1, or a pharmaceutically acceptable salt thereof, wherein the compound is: (R)-3-cyclopentyl-3-(4-(5-methyl-2-((1-methyl-1H-pyrazol-4-yl) amino) pyrimidin-4-yl)-1H-pyrazol-1-yl) propanenitrile (4);

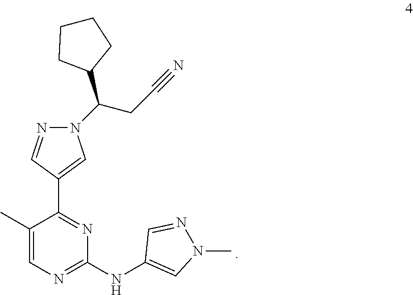

20. The compound according to claim 1, or a pharmaceutically acceptable salt thereof, wherein the compound is: (R)-3-cyclopentyl-3-(4-(5-methyl-2-((1-methyl-1H-benzo[d]imidazol-6-yl)amino)pyrimidin-4-yl)-1H-pyrazol-1-yl)propanenitrile (23);

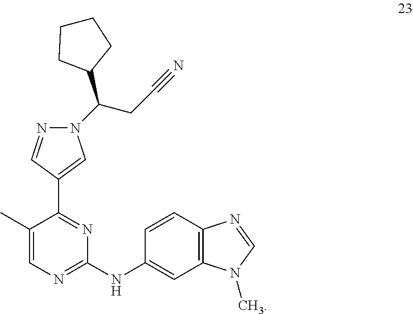 [SMILES: Cc1cnc(Nc2ccc3ncn(C)c3c2)nc1-c1cnn([C@H](CC#N)C2CCCC2)c1]

21. The compound according to claim 1, or a pharmaceutically acceptable salt thereof, wherein the compound is: (R)-3-cyclopentyl-3-(4-(2-((1-(2-hydroxyethyl)-1H-pyrazol-4-yl)amino)-5-methylpyrimidin-4-yl)-1H-pyrazol-1-yl)propanenitrile (24);

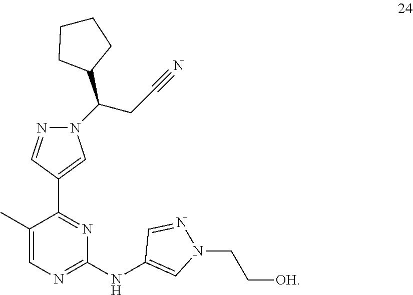 [SMILES: Cc1cnc(Nc2cnn(CCO)c2)nc1-c1cnn([C@H](CC#N)C2CCCC2)c1]

22. A pharmaceutical composition comprising a therapeutically effective amount of a compound according to of claim 1, or a pharmaceutically acceptable salt thereof together with a pharmaceutically acceptable carrier, preferably, the pharmaceutical composition is for topical administration.

23. (canceled)

24. (canceled)

25. A method for treating a dermatological disorder, the method comprising administering to a subject in need thereof a pharmaceutical composition comprising of an effective amount of a compound according to claim 1, or a salt, solvate, hydrate, polymorph, co-crystal, tautomer, stereoisomer, isotopically labeled derivative, or prodrug thereof;
preferably, the dermatological disorder can be any of those modulated by TYK2, JAK1 and JAK2;
more preferably, the dermatological disorder is atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus;
even more preferably, the dermatological disorder is atopic dermatitis, psoriasis, hives, alopecia areata or vitiligo.

26. (canceled)

27. (canceled)

28. (canceled)

29. A crystal Form A of compound (4) according to claim 19, 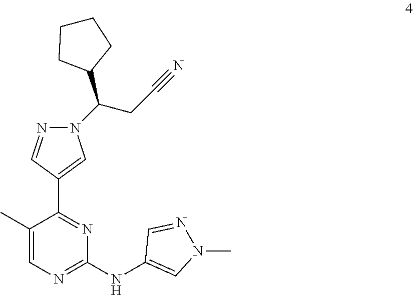
wherein the X-ray powder diffraction pattern of the crystal form shows characteristic peaks at 2theta angles of 10.3°±0.2°, 14.5°±0.2°, and 16.7°±0.2°;
preferably, wherein the X-ray powder diffraction pattern of the crystal form shows characteristic peaks at 2theta angles of 10.3°±0.2°, 14.5°±0.2°, 16.7°±0.2°, 19.8°±0.2°, 23.3°±0.2°, and 23.5°±0.2°;
more preferably, wherein the X-ray powder diffraction pattern of the crystal form shows characteristic peaks at 2theta angles of 10.3°±0.2°, 14.5°±0.2°, 16.7°±0.2°, 19.8°±0.2°, 20.7°±0.2°, 23.3°±0.2°, 23.5°±0.2°, 27.2°±0.2°, and 28.1°±0.2°.

30. (canceled)

31. (canceled)

32. The compound according to claim 2 or a pharmaceutically acceptable salt thereof,
wherein: R² is 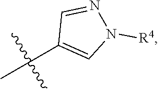
wherein R⁴ is C₁₋₃ alkyl, C₃₋₈ cycloalkyl, or 4 to 7 membered heterocyclyl, each optionally substituted with 1, 2, or 3 substituents independently selected from the group consisting of R⁵, oxo, halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′, wherein R⁵ is C₁₋₃ alkyl, or 4 to 7 membered heterocyclyl, each optionally substituted with 1, 2, or 3 substituents independently selected from the group consisting of halo, OH, CN, OR, NHR, NRR′, N(R)C(O)R′, N(R)C(O)OR′, OC(O)NRR′, C(O)R, C(O)NRR′, N(R)S(O)₂R′, S(O)₂R, and S(O)₂NRR′;
R, R′ each is independently H, or C₁₋₃ alkyl optionally substituted with 1, 2, or 3 substituents independently selected from the group consisting of halo, OH, and CN;
or, wherein: R² is 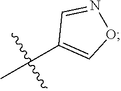 [SMILES: *c1cnoc1]
or, wherein R² is

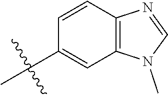 [SMILES: *c1ccc2ncn(C)c2c1]

33. The compound according to claim 2, or a pharmaceutically acceptable salt thereof, wherein: R¹ is C₁₋₃ alky, C₃₋₄ cycloalkyl, or halo.

34. The compound according to claim 2, or a pharmaceutically acceptable salt thereof, wherein: R¹ is CH₃ or Cl.

35. A method for treating a dermatological disorder, the method comprising administering to a subject in need thereof a pharmaceutical composition comprising of an effective amount of a compound according to claim 2, or a salt, solvate, hydrate, polymorph, co-crystal, tautomer, stereoisomer, isotopically labeled derivative, or prodrug thereof;
preferably, the dermatological disorder can be any of those modulated by TYK2, JAK1 and JAK2;
more preferably, the dermatological disorder is atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus;
even more preferably, the dermatological disorder is atopic dermatitis, psoriasis, hives, alopecia areata or vitiligo.

36. A method for treating a dermatological disorder, the method comprising administering to a subject in need thereof a pharmaceutical composition comprising of an effective amount of a compound according to claim 3, or a salt, solvate, hydrate, polymorph, co-crystal, tautomer, stereoisomer, isotopically labeled derivative, or prodrug thereof;
preferably, the dermatological disorder can be any of those modulated by TYK2, JAK1 and JAK2;
more preferably, the dermatological disorder is atopic dermatitis, psoriasis, hives, alopecia areata, vitiligo, hidradenitis suppurativa, hand eczema, necrobiosis lipoidica, non-sclerotic cutaneous chronic graft-versus-host disease, hand dermatitis, or Lichen planus;
even more preferably, the dermatological disorder is atopic dermatitis, psoriasis, hives, alopecia areata or vitiligo.
